(19) United States
(12) Patent Application Publication
(10) Pub. No.: US 2018/0355433 A1
(43) Pub. Date: Dec. 13, 2018

(54) CHROMOSOME NUMBER DETERMINATION METHOD

(71) Applicant: FUJIFILM Corporation, Tokyo (JP)

(72) Inventor: Takayuki TSUJIMOTO, Ashigara-kami-gun (JP)

(73) Assignee: FUJIFILM Corporation, Tokyo (JP)

(21) Appl. No.: 16/106,274

(22) Filed: Aug. 21, 2018

Related U.S. Application Data
(63) Continuation of application No. PCT/JP2017/004390, filed on Feb. 7, 2017.

(30) Foreign Application Priority Data
Feb. 24, 2016 (JP) 2016-033284

(51) Int. Cl. C12Q 1/6883; G06F 19/22

(52) U.S. Cl. CPC C12Q 1/6883; C12Q 2600/16; C12Q 2600/156; G06F 19/22

(57) ABSTRACT

Provided is a chromosome number determination method of chromosomes of interest, including: a step of performing multiplex PCR for simultaneously amplifying a plurality of loci on the chromosomes using genomic DNA extracted from a single cell or a small number of cells as templates, in which the number of loci on the chromosomes of interest is greater than or equal to 80 per chromosome, and a plurality of primer sets used in the multiplex PCR are designed through a method for designing primer sets used in a polymerase chain reaction including a first stage selection step based on a local alignment score and a second stage selection step based on a global alignment score.

### CROSS-REFERENCE TO RELATED APPLICATIONS

This application is a Continuation of PCT International Application No. PCT/JP2017/004390 filed on Feb. 7, 2017, which claims priority under 35 U.S.C. § 119(a) to Japanese Patent Application No. 2016-033284 filed on Feb. 24, 2016. The above application is hereby expressly incorporated by reference, in its entirety, into the present application.

### BACKGROUND OF THE INVENTION

### 1. Field of the Invention

The present invention relates to a chromosome number determination method.

### 2. Description of the Related Art

Genetic analysis such as deoxyribonucleic acid (DNA) base sequence analysis can be easily performed using a next generation sequencer or the like which has been developed recently. However, the total base length of a genome is generally enormous. On the other hand, there is a restriction on the reading ability of the sequencer. Therefore, in general, only a specific gene region required is amplified to limitedly read base sequences thereof. A polymerase chain reaction (PCR) method has been widespread as a technique for efficiently and precisely amplifying only a specific gene region required. Particularly, a technique for selectively amplifying a plurality of gene regions by simultaneously supplying a plurality of types of primers to one PCR reaction system is called multiplex PCR.

However, since it is difficult to directly perform PCR on a small amount of DNA such as a single cell, a region of interest is enriched through multiplex PCR and/or hybridization after amplification of the whole genome region using whole genome amplification (WGA). However, since WGA has a large amplification bias, it is difficult to accurately perform quantitative determination of the number of chromosomes.

WO2014/018080A discloses a method for reducing production of non-target amplification products generated through multiplex PCR and simultaneously amplifying a large number (one thousand to several tens of thousands) of genes to quantitatively determine chromosomes or the like. More specifically, in a case where primers are designed, an “undesirability score” between primers is designed to be less than a threshold value, and the “undesirability score” is designed so that the likelihood of formation of a primer dimer (dimer of primer) is less than or equal to a threshold value. However, there is no description of a method for specifically calculating the “undesirability score”, and it is considered that it is impossible to avoid generation of a primer dimer.

In addition, a method for designing a primer for multiplex PCR which can efficiently amplify a plurality of amplification sites (targets) is disclosed in WO2008/004691A.

### SUMMARY OF THE INVENTION

In order to improve sensitivity of the multiplex PCR itself and accurately amplify a small amount of DNA, it is conceivable to increase the number of amplified loci and increase the amount of data to be acquired. In general, however, an increase in the number of primer sets used in multiplex PCR causes formation of primer dimers and an increase in nonspecific amplification products such as amplification products from a region of non-interest. For this reason, in general multiplex PCR, even in a case where the number of amplified loci is simply increased, the quantitative determination of the number of chromosomes cannot be accurately performed from a small amount of DNA of a single cell, a small number of cells, or the like.

From the viewpoint of the above-described circumstances, an object of the present invention is to provide a chromosome number determination method of chromosomes of interest in which it is possible to accurately perform quantitative determination of the number of chromosomes from a small amount of DNA of a single cell, a small number of cells, or the like.

The present inventors have conducted extensive studies to solve the above-described problems. As a result, they have found that, in a chromosome number determination method of chromosomes of interest which includes a step of performing multiplex PCR for simultaneously amplifying a plurality of loci on the chromosomes using genomic DNA extracted from a single cell or a small number of cells as templates, in cases where the number of loci on the chromosomes of interest is greater than or equal to 80 per chromosome and a method for designing primer sets used in the multiplex PCR is a method for designing primer sets in which a local alignment score is obtained by performing pairwise local alignment on a base sequence of a primer candidate under a condition that a partial sequence to be subjected to comparison includes the 3′ terminal of the base sequence of the primer, first stage selection is performed while evaluating formability of a primer dimer based on the obtained local alignment score, a global alignment score is obtained by performing pairwise global alignment on a base sequence which has a predetermined sequence length and includes the 3′ terminal of the base sequence of the primer candidate, second stage selection is performed while evaluating formability of the primer dimer based on the obtained global alignment score, and primers selected in both of the first stage and the second stage are employed, it is possible to accurately perform quantitative determination of the number of chromosomes from a small amount of DNA of a single cell, a small number of cells, or the like, and have completed the present invention.

That is, the present invention is as [1] to [9] described below.

[1] A chromosome number determination method of chromosomes of interest, comprising: a step of performing multiplex PCR for simultaneously amplifying a plurality of loci on the chromosomes using genomic DNA extracted from a single cell or a small number of cells as templates, in which the number of loci on the chromosomes of interest is greater than or equal to 80 per chromosome, a plurality of primer sets used in the multiplex PCR are designed through a method for designing primer sets used in the polymerase chain reaction, the method for designing primer sets including a target locus selection step of selecting a target locus for designing primer sets used in the multiplex PCR from the plurality of loci, a primer candidate base sequence generation step of generating at least one base sequence of a primer candidate for amplifying the target locus regarding each of a forward-side primer and a reverse-side primer based on a base sequence in a vicinity region of the target locus on the chromosomes, a local alignment step of obtaining a local alignment score by performing pairwise local alignment on the base sequence of the primer candidate for amplifying the target locus under a condition that a partial sequence to be subjected to comparison includes the 3′ terminal of the base sequence of the primer candidate for amplifying the target locus, a first stage selection step of performing first stage selection of the base sequence of the primer candidate for amplifying the target locus based on the local alignment score obtained in the local alignment step, a global alignment step of obtaining a global alignment score by performing pairwise global alignment on a base sequence which has a predetermined sequence length and includes the 3′ terminal of the base sequence of the primer candidate for amplifying the target locus, a second stage selection step of performing second stage selection of the base sequence of the primer candidate for amplifying the target locus based on the global alignment score obtained in the global alignment step, and a primer employment step of employing the base sequence of the primer candidate which has been selected in both of the first stage selection step and the second stage selection step as the base sequence of the primer for amplifying the target locus, and both steps of the local alignment step and the first stage selection step are performed before or after both steps of the global alignment step and the second stage selection step, or performed in parallel with both steps of the global alignment step and the second stage selection step.

[2] The chromosome number determination method according to [1], in which the number of loci on the chromosomes of interest is 80 to 1,000 per chromosome.

[3] The chromosome number determination method according to [1] or [2], in which the number of loci on the chromosomes of interest is 100 to 1,000 per chromosome.

[4] The chromosome number determination method according to any one of [1] to [3], in which the number of loci on the chromosomes of interest is 100 to 500 per chromosome.

[5] The chromosome number determination method according to any one of [1] to [4], in which the chromosomes of interest contain at least one selected from the group consisting of chromosome 13, chromosome 18, and chromosome 21.

[6] The chromosome number determination method according to any one of [1] to [5], in which the steps from the target locus selection step to the primer employment step are repeated until the primer sets used in the multiplex PCR are employed for all of the plurality of loci.

[7] The chromosome number determination method according to any one of [1] to [6], in which one or more loci are selected in the target locus selection step.

[8] The chromosome number determination method according to any one of [1] to [5], in which primer sets used in the multiplex PCR are designed through a method for designing primer sets used in the polymerase chain reaction, the method for designing primer sets including a first target locus selection step of selecting a first target locus for designing primer sets used in the multiplex PCR from the plurality of loci, a first primer candidate base sequence generation step of generating at least one base sequence of a primer candidate for amplifying the first target locus regarding each of a forward-side primer and a reverse-side primer based on a base sequence in a vicinity region of the first target locus on the chromosomes, a first local alignment step of obtaining a local alignment score by performing pairwise local alignment on the base sequence of the primer candidate for amplifying the first target locus under a condition that a partial sequence to be subjected to comparison includes the 3′ terminal of the base sequence of the primer candidate for amplifying the first target locus, a first step of first stage selection of performing first stage selection of the base sequence of the primer candidate for amplifying the first target locus based on the local alignment score obtained in the first local alignment step, a first global alignment step of obtaining a global alignment score by performing pairwise global alignment on a base sequence which has a predetermined sequence length and includes the 3′ terminal of the base sequence of the primer candidate for amplifying the first target locus, a first step of second stage selection of performing second stage selection of the base sequence of the primer candidate for amplifying the first target locus based on the global alignment score obtained in the first global alignment step, a first primer employment step of employing the base sequence of the primer candidate which has been selected in both of the first step of first stage selection and the first step of second stage selection as a base sequence of a primer for amplifying the first target locus, a second target locus selection step of selecting a second target locus, which is different from the already selected target locus and in which primer sets used in the multiplex PCR are designed, from the plurality of loci, a second primer candidate base sequence generation step of generating at least one base sequence of a primer candidate for amplifying the second target locus regarding each of a forward-side primer and a reverse-side primer based on a base sequence in a vicinity region of the second target locus on the chromosomes, a second local alignment step of obtaining a local alignment score by performing pairwise local alignment on the base sequence of the primer candidate for amplifying the second target locus and the base sequence of the primer which has already been employed, under a condition that partial sequences to be subjected to comparison include the 3′ terminal of the base sequence of the primer candidate for amplifying the second target locus and the 3′ terminal of the base sequence of the primer which has already been employed, a second step of first stage selection of performing first stage selection of the base sequence of the primer candidate for amplifying the second target locus based on the local alignment score obtained in the second local alignment step, a second global alignment step of obtaining a global alignment score by performing pairwise global alignment on base sequences which have a predetermined sequence length and include the 3′ terminal of the base sequence of the primer candidate for amplifying the second target locus and the 3′ terminal of the base sequence of the primer which has already been employed, a second step of second stage selection of performing second stage selection of the base sequence of the primer candidate for amplifying the second target locus based on the global alignment score obtained in the second global alignment step, and a second primer employment step of employing the base sequence of the primer candidate which has been selected in both of the second step of first stage selection and the second step of second stage selection as a base sequence of a primer for amplifying the second target locus, both steps of the first local alignment step and the first step of first stage selection are performed before or after both steps of the first global alignment step and the first step of second stage selection, or performed in parallel with both steps of the first global alignment step and the first step of second stage selection, both steps of the second local alignment step and the second step of first stage selection are performed before or after both steps of the second global alignment step and the second step of second stage selection, or performed in parallel with both steps of the second global alignment step and the second step of second stage selection, and in a case where the number of the plurality of loci is three or more, the steps from the second target locus selection step to the second primer employment step are repeated until the primer sets used in the multiplex PCR are employed for all of the plurality of loci.

[9] The chromosome number determination method according to any one of [1] to [8], in which the templates are not amplification products obtained through whole genome amplification of genomic DNA.

According to the present invention, it is possible to provide a chromosome number determination method of chromosomes of interest in which it is possible to accurately perform quantitative determination of the number of chromosomes which are objects of the quantitative determination of the number of chromosomes from a small amount of DNA of a single cell, a small number of cells, or the like.

In addition, according to the chromosome number determination method of the present invention, the method is not performed through whole genome amplification (WGA), and therefore, it is possible to eliminate bias caused by WGA in the related art.

### BRIEF DESCRIPTION OF THE DRAWINGS

FIG. 1 is a block diagram representing a method for designing primer sets in the present invention.

FIG. 2 is a diagram showing local alignment, a local alignment score, global alignment, and a global alignment score of a base sequence of SEQ ID No: 1 and a base sequence of SEQ ID No: 2.

FIG. 3 is a diagram showing local alignment, a local alignment score, global alignment, and a global alignment score of a base sequence of SEQ ID No: 21 and a base sequence of SEQ ID No: 22.

FIG. 4 is a diagram showing local alignment, a local alignment score, global alignment, and a global alignment score of a base sequence of SEQ ID No: 41 and a base sequence of SEQ ID No: 42.

FIG. 5 is a diagram showing local alignment, a local alignment score, global alignment, and a global alignment score of a base sequence of SEQ ID No: 61 and a base sequence of SEQ ID No: 62.

FIG. 6 is a diagram showing local alignment, a local alignment score, global alignment, and a global alignment score of a base sequence of SEQ ID No: 81 and a base sequence of SEQ ID No: 82.

FIG. 7 is a graph showing a relationship between the (total) number of loci and a coefficient of variation which are derived from results of an example and comparative examples. Plots and an approximate curve which represent data obtained from the example and the comparative examples are shown in the graph.

### DESCRIPTION OF THE PREFERRED EMBODIMENTS

Hereinafter, a chromosome number determination method of chromosomes of interest of the present invention will be described in detail.

In the present specification, the range represented by “to” means a range including both ends denoted before and after “to”.

[Step of Performing Multiplex PCR]

The step of performing multiplex PCR includes a step of performing multiplex PCR for simultaneously amplifying a plurality of loci on chromosomes, in which loci to be amplified exist, using genomic DNA extracted from a single cell or a small number of cells as templates.

<Genomic DNA Extracted from Single Cell or Small Number of Cells>

Genomic DNA extracted from a single cell or a small number of cells will be described below.

The “single cell” refers to one cell and a “small number of cells” refers to a number of cells of less than 10.

The genomic DNA refers to DNA extracted from a cell. Although the genomic DNA may be concentrated or diluted, a whole genome amplification product which is obtained by amplifying genomic DNA through whole genome amplification and a specific region amplification product obtained by amplifying a specific region of genomic DNA are not included in the genomic DNA.

«Genomic DNA Extracted from Single Cell»

A genomic DNA extracted from a single cell can be prepared, for example, by isolating a single cell from a population of cells and extracting the genomic DNA from the isolated single cell.

The method for isolating a single cell from a population of cells is not particularly limited, and a well-known method in the related art can be used. A method for isolating a single cell from a maternal blood sample will be described as an example. Even for samples other than the maternal blood sample, a method described below can be appropriately modified and used.

(Maternal Blood Sample)

The maternal blood sample is not particularly limited as long as the sample is a blood sample collected from a maternal body (pregnant woman), and maternal peripheral blood is preferable. Maternal body-derived nucleated red blood cells and fetus-derived nucleated red blood cells are included in the maternal peripheral blood in addition to white blood cells such as maternal body-derived eosinophils, neutrophils, basophils, mononuclear cells, and lymphocytes, and mature red blood cells having no nucleus. It has been known that fetus-derived nucleated red blood cells exist in maternal blood from about 6 weeks after pregnancy. For this reason, in the present invention, it is preferable to test peripheral blood of a pregnant woman after about 6 weeks of pregnancy.

(Fetal Nucleated Red Blood Cell)

The single cell is not particularly limited as long as the single cell is derived from a fetus, but a fetus-derived nucleated red blood cell is preferable. The fetus-derived nucleated red blood cell is a red blood cell precursor existing in maternal blood. During pregnancy of a mother, a red blood cell of a fetus may be nucleated. Since there is a chromosome in this red blood cell, a fetus-derived chromosome and a fetal gene become available using less invasive means. It has been known that this fetus-derived nucleated red blood cell exists at a rate of one in 10⁶ cells in the maternal blood, and the existence probability of the fetus-derived nucleated red blood cell in peripheral blood in a pregnant woman is extremely low.

(Concentration of Fetal Nucleated Red Blood Cell)

Fetus-derived nucleated red blood cells can be concentrated through density gradient centrifugation as a preferred embodiment in a case of isolating single cells.

The density of blood cells in a maternal body including fetus-derived nucleated red blood cells is disclosed in WO2012/023298A. According to the disclosure, the assumed density of the fetus-derived nucleated red blood cells is about 1.065 to 1.095 g/mL. On the other hand, the density of blood cells of the maternal blood is about 1.070 to 1.120 g/mL in a case of red blood cells, about 1.090 to 1.110 g/mL in a case of eosinophils, about 1.075 to 1.100 g/mL in a case of neutrophils, about 1.070 to 1.080 g/mL in a case of basophils, about 1.060 to 1.080 g/mL in a case of lymphocytes, and about 1.060 to 1.070 g/mL in a case of mononuclear cells.

In a case where fetus-derived nucleated red blood cells are concentrated through density gradient centrifugation, it is possible to use media such as Percoll (manufactured by GE Healthcare Bioscience) that is a silicic acid colloidal particle dispersion which is coated with polyvinylpyrrolidone and has a diameter of 15 to 30 nm, Ficoll-Paque (manufactured by GE Healthcare Bioscience) which is a neutral hydrophilic polymer which is rich in side chains and formed of sucrose, and/or Histopaque (manufactured by Sigma-Aldrich Co. LLC.) which is a solution using polysucrose and sodium diatrizoate, as a first medium and a second medium.

In the present invention, it is preferable to use Percoll and/or Histopaque. A product with a density of 1.130 g/cm³ (specific gravity of 1.130) is commercially available as Percoll, and it is possible to prepare a medium with a target density (specific gravity) by diluting the product. In addition, a medium with a density of 1.077 g/cm³ (specific gravity of 1.077) and a medium with a density of 1.119 g/cm³ (specific gravity of 1.119) are commercially available as Histopaque, and it is possible to prepare a medium with a target density (specific gravity) by mixing these media with each other. By using Percoll and Histopaque, it is possible to prepare a first medium and a second medium.

The density of media to be stacked is set in order to separate fetus-derived nucleated red blood cells having a density of about 1.065 to 1.095 g/mL from other blood cell components in a maternal body. The central density of fetus-derived nucleated red blood cells is about 1.080 g/mL. Therefore, in a case where two media (first medium and second medium) having different densities interposing the central density are prepared and are made to be adjacent to and overlap each other, it is possible to collect fractions having the desired fetus-derived nucleated red blood cells on an interface between the media. It is preferable that the density of the first medium is set to be 1.080 g/mL to 1.100 g/mL and the density of the second medium is set to be 1.060 g/mL to 1.080 g/mL. It is more preferable that the density of the first medium is set to be 1.080 g/mL to 1.090 g/mL and the density of the second medium is set to be 1.065 g/mL to 1.080 g/mL. As a specific embodiment, it is preferable to separate plasma components, eosinophils, and mononuclear cells from the desired fractions to be collected, by setting the density of the first medium to 1.085 g/mL and the density of the second medium to 1.075 g/mL. In addition, by setting the densities of the media, it is also possible to partially separate red blood cells, neutrophils, and lymphocytes therefrom. In the present invention, the type of the first medium and the type of the second medium may be the same as or different from each other. However, the types of the media are preferably the same as each other.

(Sorting and Isolating of Nucleated Red Blood Cell Candidate)

Examples of a method of isolating a single cell include a method for peeling cells one by one from a transparent substrate with a micromanipulator, and sorting performed through immunological dyeing and fluorescence activated cell sorting (FACS).

Hereinafter, the method for peeling a single cell from a transparent substrate with a micromanipulator will be described in detail.

In order to obtain a nucleated red blood cell candidate from maternal blood, it is possible to prepare a substrate (blood cell specimen) coated with blood cells by coating the top of the substrate with blood and drying the blood. A transparent medium is preferably used as this substrate and slide glass is more preferably used as this substrate.

It is possible to sort out a fetus-derived nucleated red blood cell candidate based on the information on the shape of blood cells obtained from the blood cell specimen. As a preferred embodiment, it is possible to sort out a fetus-derived nucleated red blood cell candidate using a ratio of the area of a nuclear region to the area of cytoplasm of a cell, the degree of circularity of a nucleus, and/or the area of a nuclear region, and the like. Particularly, it is preferable to sort out a cell in which the ratio of the area of a nuclear region to the area of cytoplasm or the degree of circularity of a nucleus satisfies the conditions, as a fetus-derived nucleated red blood cell candidate.

In the present invention, it is preferable to sort out cells in which the ratio “N/C” of the area of a nuclear region to the area of cytoplasm satisfies Formula (1).

0.25<N/C<1.0 (1)

However, in Formula (1), “N” represents the area of a nuclear region of a cell on which image analysis is to be performed and “C” represents the area of cytoplasm of a cell on which image analysis is to be performed.

In addition, in the present invention, it is preferable to sort out cells in which the ratio “N/L²” of the area of the nuclear region to the square of the length of the major axis of a nucleus satisfies Formula (2).

0.65<N/L²<0.785 (2)

However, in Formula (2), “N” represents the area of a nuclear region of a cell on which image analysis is to be performed and “L” represents the length of a major axis of a nucleus of a cell on which image analysis is to be performed, that is, the length of a major axis of an ellipse circumscribing a cell nucleus which has a complicated shape.

A system of sorting out a fetus-derived nucleated red blood cell candidate using information on the shape of cells is equipped with an optical microscope, a digital camera, a stage for slide glass, an optical transfer system, an image processing PC, a control PC, and a display. The optical transfer system includes an objective lens and a CCD camera. The image processing PC includes a processing system of performing data analysis and storing of data. The control PC includes a control system of controlling the position of a stage for slide glass or controlling the entire processing.

A protein existing in a red blood cell in the blood of all vertebrates including human beings is hemoglobin. The presence or absence of hemoglobin in a nucleated red blood cell is different from the presence or absence of hemoglobin in a white blood cell which is a type of nucleated cell in blood. Hemoglobin in a case of being bonded to oxygen is oxygenated hemoglobin exhibiting clear red color, and hemoglobin in a case of not being bonded to oxygen is reduced hemoglobin exhibiting dark red color. Hemoglobin having different oxygen bonding amounts flows in the arteries and the veins. Hemoglobin has absorption at 380 nm to 650 nm. Therefore, it is possible to detect hemoglobin using information of at least one monochromatic light beam caused by the difference in the absorbance of this wavelength range. It is preferable to use monochromatic light in order to check the presence or absence of hemoglobin. It is possible to select light with a single wavelength in a wavelength range of 400 nm to 500 nm or monochromatic light in a wavelength range of 525 nm to 580 nm, in which the absorbance of hemoglobin is large. The absorption coefficients of these wavelength ranges show high values due to the existence of hemoglobin. Therefore, the ratio of each absorption coefficient of these wavelength ranges to the absorption coefficient of cytoplasm of a white blood cell becomes greater than or equal to 1.

As an embodiment, it is possible to identify a cell in which a cell nucleus having a nearly circular shape exists and which has hemoglobin, as a nucleated red blood cell candidate. Furthermore, in fetus-derived nucleated red blood cells and adult-derived nucleated red blood cells, hemoglobin of a fetus is hemoglobin F (HbF) and hemoglobin of an adult is hemoglobin A (HbA). Therefore, it is possible to sort out fetus-derived nucleated red blood cells using the difference in spectral characteristics caused by different oxygen bonding abilities.

In a case of measuring the absorption coefficient of cytoplasm, it is possible to use a microspectrophotometer. The microspectrophotometer is a photometer in which the same principle as that of a usual spectrophotometer is used for an optical system of a microscope, and it is possible to use a commercially available device.

In some cases, it is impossible to define whether the isolated nucleated red blood cell is derived from a fetus or from a maternal body (pregnant woman) depending on only the information on the shape and/or the absorbance of the cell. However, in the present invention, it is possible to discriminate the origin of the isolated nucleated red blood cell through polymorphism analysis using SNP and/or short tandem repeat (STR: short tandem repeat sequence) or the like, and through DNA analysis such as checking the presence of a Y chromosome.

(Extraction of Genomic DNA)

Extraction of genomic DNA from a single cell can be performed through a well-known method in the related art. It is preferable to use a commercially available DNA extraction kit. Examples of a commercially available DNA extraction kit that can be used for genomic DNA extraction from a single cell include Single Cell WGA Kit (manufactured by New England Biolabs). In a case where the commercially available DNA extraction kit is used, DNA extraction may be carried out according to the protocol attached to the kit, but the protocol may be appropriately modified and used.

«Genomic DNA Extracted from Small Number of Cells»

Genomic DNA extracted from a small number of cells can be prepared, for example, by separating a small number of cells from a population of cells, extracting genomic DNA from the small number of isolated cells, by isolating single cells from a population of cells, mixing the isolated single cells with each other, and extracting genomic DNA from a small number of the mixed cells, by isolating a single cell from a population of cells, extracting genomic DNA from the isolated single cell, and mixing the extracted genomic DNA's with each other, or by a combination of two or more of these methods.

<Multiplex PCR>

Multiplex PCR is PCR for simultaneously amplifying a plurality of loci on chromosomes using a plurality of primer sets.

«Thermal Cycle»

Multiplex PCR includes a plurality of thermal cycles including thermal denaturation, annealing, and elongation. The multiplex PCR may further include initial thermal denaturation and/or final elongation as desired.

(Thermal Denaturation)

The conditions of thermal denaturation such as the temperature and the time are not particularly limited as long as it is possible to dissociate two chains of genomic DNA to make a chain.

As examples of suitable conditions for thermal denaturation, the temperature is set to 90° C. to 95° C. and preferably to 94° C.±2° C. and the time is set to 10 seconds to 60 seconds and preferably 30 seconds ±5 seconds.

The temperature and time of thermal denaturation may be appropriately changed depending on the amount of genomic DNA of templates.

(Annealing)

The conditions of annealing such as the temperature and the time are not particularly limited as long as it is possible to bond a primer to genomic DNA which has been disassociated to become a chain.

As examples of suitable conditions for annealing, the temperature is set to 50° C. to 65° C. and preferably to 60° C.±2° C. and the time is set to 10 seconds to 90 seconds and preferably 60±10 seconds.

The temperature and time of annealing may be appropriately changed depending on the GC content (referring to a total mole percentage of guanine (abbreviation=G) and cytosine (abbreviation=C) in all nucleic acid bases) of a primer, a Tm value (which is a temperature at which 50% of double-stranded DNA is dissociated and becomes single-stranded DNA and in which Tm is derived from a melting temperature), and deviation of a sequence.

(Elongation)

The elongation condition is not particularly limited as long as it is a temperature and time at which the polynucleotide chain can be elongated from the 3′ terminal of a primer by DNA polymerase.

As examples of suitable conditions for elongation, the temperature is set to 72° C.±2° C. and the time is set to 10 seconds to 60 seconds and preferably 30±5 seconds.

The temperature and time for elongation may be appropriately changed depending on the type of DNA polymerase and/or the size of PCR amplification product.

(Initial Thermal Denaturation)

Initial thermal denaturation may be performed before the first cycle of a thermal cycle is started.

The conditions of initial thermal denaturation may be the same as or different from the conditions of the thermal denaturation. In the case where the conditions of initial thermal denaturation are different from the conditions of the thermal denaturation, it is preferable to set the temperature at the same temperature as in the case of the thermal denaturation and set the time to be longer than that of the thermal denaturation.

By performing the initial thermal denaturation, it is possible to more reliably dissociate two chains of genomic DNA in the first cycle of the thermal cycle.

(Final Elongation)

Final elongation may be performed after the last cycle of the thermal cycle is completed.

The conditions of final elongation may be the same as or different from the conditions of the elongation. In the case where the conditions of final elongation are different from the conditions of the elongation, it is preferable to set the temperature at the same temperature as in the case of the thermal denaturation and set the time to be longer than that of the elongation.

By performing the final elongation, it is possible to more reliably elongate a polynucleotide chain.

(Number of Cycles)

The number of cycles is not particularly limited as long as it is plural, but is preferably 20 cycles to 40 cycles and more preferably 35±5 cycles.

The number of cycles may be appropriately changed depending on the amount of genomic DNA which becomes a template of multiplex PCR, the number of primer sets used for the multiplex PCR, and/or the amount of reaction solution of multiplex PCR.

Although the amplification product obtained through PCR theoretically increases twice per cycle, in reality, it reaches a plateau in a certain cycle, and there is a possibility that amplification products more than that may not be desired or a nonspecific amplification product may increase. Therefore, it cannot be said that it is desirable to increase the number of cycles unconditionally.

(Primer Set)

A primer set designed according to “Method for Designing Primer Sets” to be described below can be used as a primer set used in multiplex PCR, Since the primer set is designed not to form a primer dimer, it is possible to suppress the increase in the nonspecific amplification product and to improve the sensitivity of the multiplex PCR itself.

The number of primer sets is set corresponding to the number of loci to be amplified. An identical locus may be amplified by two or more pairs of primer sets, or two or more loci may be amplified by a pair of primer sets. In general, it is preferable that the locus corresponds one to one to the primer set.

«Plurality of Loci on Chromosomes»

(Chromosomes)

Chromosomes include one or more selected from the group consisting of an autosome from chromosome 1 to chromosome 22 of a human and a sex chromosome of X and Y chromosomes.

The chromosomes are not particularly limited as long as these include chromosomes (in the present invention, in particular, there are some cases in which these may be referred to as “chromosomes of interest”) to be subjected to quantitative determination of the number of chromosomes.

The chromosomes may include a chromosome that provides a reference value for quantitative determination of chromosomes and/or a chromosome that is only interested in the presence or absence of loci, in addition to the chromosomes to be subjected to quantitative determination of the number of chromosomes. That is, even in a case of chromosomes in which loci to be amplified through multiplex PCR exist, the chromosome that provides a reference value for quantitative determination of chromosomes and/or a chromosome that is only interested in the presence or absence of loci are excluded from the chromosomes (chromosomes of interest) to be subjected to quantitative determination of the number of chromosomes.

The chromosomes of interest particularly preferably contain at least one selected from the group consisting of chromosome 13, chromosome 18 and chromosome 21. These chromosomes are more likely to generate trisomy or monosomy compared to other autosomes.

Examples of the chromosome that provides a reference value for quantitative determination of chromosomes include an autosome and/or an X chromosome other than the chromosomes which are likely to generate trisomy or monosomy. Among them, the X chromosome is a preferred chromosome because it exists regardless of the gender of men and women.

An example of the chromosome that is only interested in the presence or absence of loci includes a Y chromosome. This is because the presence of the Y chromosome strongly suggests male among the genders of men and women. In a case of quantitatively determining the number of chromosomes of a fetus, it is preferable that the chromosomes include a Y chromosome in order to discriminate cells derived from a mother (maternal boy) from cells derived from a fetus. This is because the presence of the Y chromosome suggests a denial of the origin of cells derived from a mother (maternal body).

(Plurality of Loci)

A plurality of loci are loci to be amplified through multiplex PCR out of loci on chromosomes.

Since the chromosomes may include chromosome that provides a reference value for quantitative determination of chromosomes and/or a chromosome that is only interested in the presence or absence of loci in addition to the chromosomes (chromosomes of interest) to be subjected to quantitative determination of the number of chromosomes, the plurality of loci are not limited to those existing on the chromosomes to be subjected to quantitative determination of the number of chromosomes and may include loci existing on the chromosome that provides a reference value for quantitative determination of chromosomes and/or loci existing on the chromosome that is only interested in the presence or absence of loci.

The loci may exist in either a gene region or a non-gene region.

The gene region includes: a coding region in which a gene encoding proteins, a ribosomal ribonucleic acid (RNA) gene, a transfer RNA gene, and the like exist; and a non-coding region in which an intron dividing a gene, a transcription regulatory region, a 5′ leader sequence, a 3′ trailer sequence, and the like exist.

The non-gene region includes: a non-repetitive sequence such as a pseudogene, a spacer, a response element, and a replication origin; and a repetitive sequence such as a tandem repetitive sequence and an interspersed repetitive sequence.

Loci may be, for example, loci such as single nucleotide polymorphism (SNP), single nucleotide variant (SNV), short tandem repeat polymorphism (STRP), mutation, and insertion and/or deletion (indel).

(Number of Loci)

The number of loci on the chromosomes (chromosomes of interest) to be subjected to quantitative determination of the number of chromosomes is not particularly limited as long as it is greater than or equal to 80 and the chromosomes are chromosomes of interest, but is preferably 80 to 1,000, more preferably 100 to 1,000, still more preferably 100 to 500, and still more preferably 100 to 200. In particular, the number of loci per chromosome is preferably 150 to 200.

In a case where the number of loci per chromosome of interest is within this ranges, the coefficient of variation of the coverage becomes sufficiently small, and therefore, the accuracy of the quantitative determination of the number of chromosomes can be improved.

[Step of Quantitatively Determining Number of Chromosomes]

In the present invention, the quantitative determination of the number of chromosomes can be carried out through a well-known method in the related art, but is preferably carried out, for example, through a method to be described below using a next generation sequencer.

It is desirable to particularly use Miseq (manufactured by Illumina, Inc.) as the next generation sequencer. In a case of sequencing a plurality of multiplex PCR amplification products using the next generation sequencer “Miseq”, it is necessary to add P5 and P7 sequences, which are used for hybridizing to a sample identification sequence (index sequence) formed of 6 to 8 bases, and an oligonucleotide sequence on the top of a Miseq flow cell, to each of the multiplex PCR amplification products. By adding these sequences thereto, it is possible to measure up to 96 types of multiplex PCR amplification products at a time.

It is possible to use an adapter ligation method or a PCR method as the method for adding an index sequence and P5 and P7 sequences to both terminals of the multiplex PCR amplification products.

As the method for analyzing sequence data obtained using Miseq to quantitatively determine the number of chromosomes, it is preferable to map the sequence data in a well-known human genome sequence using Burrows-Wheeler Aligner (BWA: Li, H., et al., “Fast and accurate short read alignment with Burrows-Wheeler transform”, Bioinformatics, 2009, Vol. 25, No. 14, PP. 1754-1760; and Li, H., et al., “Fast and accurate long-read alignment with Burrows-Wheeler transform”, Bioinformatics, 2010, Vol. 26, No. 5, PP. 589-595). As means for analyzing a genetic abnormality, it is preferable to quantitatively determine the number of chromosomes using SAMtools (Li, Heng, et al., “The Sequence Alignment/Map format and SAMtools”, Bioinformatics, 2009, Vol. 25, No. 16, PP. 2078-2079; SAM is derived from “Sequence Alignment/Map”) and/or BEDtools (Quinlan, A. R., et al., “BEDtools: a flexible suite of utilities for comparing genomic features”, Bioinformatics, 2010, Vol. 26, No. 6, PP. 841-842).

For example, regarding DNA fragments in which fetal nucleated red blood cells are identified and which are obtained by performing PCR amplification of a target locus, the amplification amount (the coverage, the sequence depth, and the number of times of sequence reading) of amplification product having a sequence of a region of 140 bp to 180 bp which has been previously determined can be obtained using a sequencer.

Regarding a cell which has been identified as a mother-derived nucleated red blood cell, the amplification amount (number of times of sequence reading) of amplification product having a sequence of a region of 140 bp to 180 bp which has been previously determined is obtained as a standard (reference) using the sequencer. In a case where fetuses are in normal states, it is expected that the ratio of the amplification amount (number of times of sequence reading) of mother-derived amplification product to the amplification amount (number of times of sequence reading) of fetus-derived amplification product becomes almost 1:1. In a case where fetuses have a disease which is trisomy derived from an amplified chromosome, it is expected that the ratio thereof becomes almost 1:1.5 (or 2:3).

In the present invention, the proportions of the amount (number of times of sequence reading) of fetus-derived PCR amplification products to the amount (number of times of sequence reading) of mother-derived PCR amplification products which have been collected from a plurality of pregnant maternal bodies in a case where the mothers are pregnant with normal fetuses are obtained plural times, and the distribution thereof is obtained. In addition, the proportions of the amount (number of times of sequence reading) of fetus-derived amplification products to the amount (number of times of sequence reading) of mother-derived amplification products in a case where the mothers are pregnant with fetuses with trisomy are obtained, and the distribution thereof is obtained. It is also possible to set a cutoff value in a region where these two distributions do not overlap. After comparing the cutoff value which has previously been determined with a result in which the proportion of the amplification products is obtained, it is also possible to interpret inspection results that the fetuses are normal in a case where the proportion thereof is less than or equal to the cutoff value, and the fetuses have trisomy in a case where the proportion thereof is greater than or equal to the cutoff value.

[Method for Designing Primer Sets]

Hereinafter, the method for designing primer sets which is one of the characteristic features of the present invention will be described in detail.

A first embodiment of the method for designing primer sets in the present invention includes the following steps:
(a) a target locus selection step of selecting a target locus for designing primer sets used in the multiplex PCR from the plurality of loci;
(b) a primer candidate base sequence generation step of generating at least one base sequence of a primer candidate for amplifying the target locus regarding each of a forward-side primer and a reverse-side primer based on a base sequence in a vicinity region of the target locus on the chromosomes;
(c) a local alignment step of obtaining a local alignment score by performing pairwise local alignment on the base sequence of the primer candidate for amplifying the target locus under a condition that a partial sequence to be subjected to comparison includes the 3′ terminal of the base sequence of the primer candidate for amplifying the target locus;
(d) a first stage selection step of performing first stage selection of the base sequence of the primer candidate for amplifying the target locus based on the local alignment score obtained in the local alignment step;
(e) a global alignment step of obtaining a global alignment score by performing pairwise global alignment on a base sequence which has a predetermined sequence length and includes the 3′ terminal of the base sequence of the primer candidate for amplifying the target locus;
(f) a second stage selection step of performing second stage selection of the base sequence of the primer candidate for amplifying the target locus based on the global alignment score obtained in the global alignment step; and
(g) a primer employment step of employing the base sequence of the primer candidate which has been selected in both of the first stage selection step and the second stage selection step as the base sequence of the primer for amplifying the target locus.

However, both steps of (c) Local Alignment Step and (d) First Stage Selection Step are performed before or after both steps of (e) Global Alignment Step and (f) Second Stage Selection Step, or performed in parallel with both steps of (e) Global Alignment Step and (f) Second Stage Selection Step.

Each step of the first embodiment of the method for designing primer sets in the present invention will be described in detail.

(a) Target Locus Selection Step

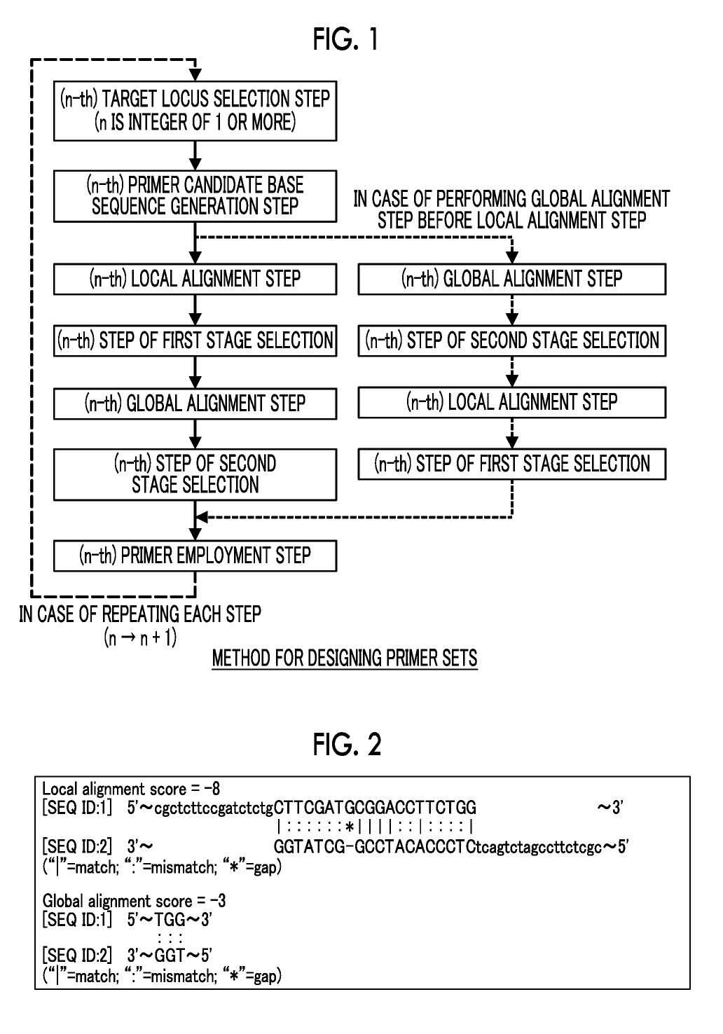

(a) Target Locus Selection Step is shown in the block diagram of FIG. 1 as “(n-th) TARGET LOCUS SELECTION STEP”.

The target locus selection step is a step of selecting a locus (target locus) primer for designing sets used in the multiplex PCR from the plurality of loci.

The number of a plurality of loci to be amplified through multiplex PCR is N (where N is an integer satisfying N≥2) and the number of target loci that can be selected is n (n is an integer satisfying 1≤n≤N).

In a case where two or more loci are selected, successive primer sets may be designed for each locus, primer sets may be designed in parallel for each locus, or primer sets may be designed at the same time for each locus.

(b) Primer Candidate Base Sequence Generation Step

(b) Primer Candidate Base Sequence Generation Step is shown in the block diagram of FIG. 1 as “(n-th) PRIMER CANDIDATE BASE SEQUENCE GENERATION STEP”.

The primer candidate base sequence generation step is a step of generating at least one base sequence of a primer candidate for amplifying the target locus regarding each of a forward-side primer and a reverse-side primer based on a base sequence in a vicinity region of the target locus on the chromosomes.

A base sequence of a primer candidate is generated based on the base sequence of the above-described vicinity region, but may have a portion not complementary to the base sequence of the above-described vicinity region at a 5′ terminal side. In some cases, such a portion not complementary to the 5′ terminal side of the primer may be used to add a specific base sequence to an amplification product obtained through multiplex PCR.

The vicinity region of a target locus is a region excluding the target locus in a region including the target locus on a chromosome.

The length of a vicinity region is not particularly limited, but is preferably less than or equal to a length that can be expanded through PCR and more preferably less than or equal to the upper limit of a fragment length of DNA for which amplification is desired. A length facilitating application of concentration selection and/or sequence reading is particularly preferable. The length of a vicinity region may be appropriately changed in accordance with the type of enzyme (DNA polymerase) used for PCR. The specific length of a vicinity region is preferably about 20 to 500 bases, more preferably about 20 to 300 bases, still more preferably about 20 to 200 bases, and particularly preferably about 50 to 200 bases.

In addition, in a case of generating a base sequence of a primer candidate, points, such as the length of a complementary portion of a primer, the total length of a primer, the GC content (referring to a total mole percentage of guanine (abbreviation=G) and cytosine (abbreviation=C) in all nucleic acid bases), a Tm value (which is a temperature at which 50% of double-stranded DNA is dissociated and becomes single-stranded DNA and in which Tm is derived from a melting temperature), and deviation of a sequence, to be taken into consideration in a general method for designing a primer are the same.

The complementary portion of a primer is a portion at which the primer hybridizes to single-stranded DNA of a template during annealing. A base sequence of a complementary portion of a primer is generated based on a base sequence of a vicinity region of a target locus. In the present invention, a non-complementary portion may be linked to the 5′ terminal of the complementary portion of the primer. The non-complementary portion is a portion at which the primer is not intended to hybridize to single-stranded DNA of template DNA. As the base sequence of the non-complementary portion of the primer, there is a tail sequence used for adding a sequence for sequencing to an amplification product, which is obtained through multiplex PCR, by performing PCR (second PCR) using the amplification product as a template.

The length of a complementary portion of a primer (the number of nucleotides) is not particularly limited, but is preferably 10 mer to 30 mer, more preferably 15 mer to 30 mer, and still more preferably 15 mer to 25 mer. In a case where the length of a complementary portion of a primer is within this range, it is easy to design a primer excellent in specificity and amplification efficiency.

The GC content is not particularly limited, but is preferably 40 mol % to 60 mol % and more preferably 45 mol % to 55 mol %. In a case where the GC content is within this range, a problem such as a decrease in the specificity and the amplification efficiency due to a high-order structure is less likely to occur.

The Tm value is not particularly limited, but is preferably within a range of 50° C. to 65° C. and more preferably within a range of 55° C. to 65° C.

The Tm value can be calculated using software such as OLIGO Primer Analysis Software (manufactured by Molecular Biology Insights) or Primer 3 (http://www-genome.wi.mit.edu/ftp/distribution/software/).

In addition, the Tm value can also be obtained through calculation using the following formula from the number of A's, T's, G's, and C's (which are respectively set as nA, nT, nG, and nC) in a base sequence of a primer.

Tm value (° C.)=2(nA+nT)+4(nC+nG)

The method for calculating the Tm value is not limited thereto and can be calculated through various well-known methods in the related art.

The base sequence of a primer candidate is preferably set as a sequence in which there is no deviation of bases as a whole. For example, it is desirable to avoid a GC-rich sequence and a partial AT-rich sequence.

In addition, it is also desirable to avoid continuation of T and/or C (polypyrimidine) and continuation of A and/or G (polypurine).

Furthermore, it is preferable that a 3′ terminal base sequence avoids a GC-rich sequence or an AT-rich sequence. G or C is preferable for a 3′ terminal base, but is not limited thereto.

(Specificity-Checking Step)

As desired, a specificity-checking step may be performed.

The specificity-checking step is a step of evaluating specificity of a base sequence of a primer candidate may be performed based on sequence complementarity with respect to genomic DNA of a base sequence of each primer candidate which has been generated in (b) Primer Candidate Base Sequence Generation Step.

In the specificity check, in a case where local alignment of a base sequence of genomic DNA and a base sequence of a primer candidate is performed and a local alignment score is less than a predetermined value, it is possible to evaluate that the complementarity of the base sequence of the primer candidate with respect to genomic DNA is low and the specificity of the base sequence of the primer candidate with respect to genomic DNA is high. Here, it is desirable to perform local alignment on also a complementary chain of genomic DNA. This is because genomic DNA is double-stranded whereas the primer is single-stranded DNA. In addition, a base sequence complementary to the base sequence of the primer candidate may be used instead of the base sequence of the primer candidate. The complementarity can be considered as homology with respect to a complementary chain.

In addition, homology search may be performed on genomic DNA base sequence database using the base sequence of the primer candidate as a query sequence. Examples of a homology search tool include Basic Local Alignment Search Tool (BLAST) (Altschul, S. A., et al., “Basic Local Alignment Search Tool”, Journal of Molecular Biology, 1990, October, Vol. 215, pp. 403-410) and FASTA (Pearson, W. R., et al., “Improved tools for biological sequence comparison”, Proceedings of the National Academy of Sciences of the United States of America, National Academy of Sciences, 1988, April, Vol. 85, pp. 2444-2448). It is possible to obtain local alignment as a result of performing the homology search.

Scores given to each of a complementary base (match), a non-complementary base (mismatch) and a gap (insertion and/or deletion (indel)) (in some cases, referred to as a “scoring system” in the present specification), and a threshold value of a local alignment score are not particularly limited, and can be appropriately set depending on the length of a base sequence of a primer candidate and/or the PCR conditions. In a case of using a homology search tool, a default value of the homology search tool may be used.

For example, as the scoring system, it is considered that complementary base (match)=+1, non-complementary base (mismatch)=−1, and gap (insertion and/or deletion (indel))=−3 are employed and the threshold value is set to be +15. In some cases, the score for the gap is referred to as a gap penalty.

In a case where a base sequence of a primer candidate has complementarity to a base sequence at an unexpected position on genomic DNA but has low specificity thereto, in some cases, an artifact is amplified instead of amplifying a target locus in a case where PCR is performed using a primer of the base sequence of a primer candidate. Therefore, the case where the base sequence of the primer candidate has complementarity to the base sequence at an unexpected position on genomic DNA but has low specificity thereto is excluded.

(c) Local Alignment Step

(c) Local Alignment Step is shown in the block diagram of FIG. 1 as “(n-th) LOCAL ALIGNMENT STEP”.

The local alignment step is a step of obtaining a local alignment score by performing pairwise local alignment on the base sequence of the primer candidate for amplifying the target locus under a condition that a partial sequence to be subjected to comparison includes the 3′ terminal of the base sequence of the primer candidate for amplifying the target locus.

A combination of pairs of base sequences to be subjected to local alignment may be a combination selected while allowing overlapping, or may be a combination selected without allowing overlapping. However, in a case where formability of a primer dimer between primers of an identical base sequence has not yet been evaluated, the combination selected while allowing overlapping is preferable.

The total number of combinations is “ₘH₂=ₘ₊₁C₂=(m+1)!/2(m−1)!” in a case where the selection is performed while allowing overlapping, and is “ₘC₂=m(m−1)/2” in a case where the selection is performed without allowing overlapping, in which the number of base sequences which have been generated in (b) Primer Candidate Base Sequence Generation Step is set to be m.

In a case where both steps of (e) Global Alignment Step and (f) Second Stage Selection Step to be described below are performed first, the present step and (d) First Stage Selection Step to be described below may be performed on primer candidates selected in (f) Second Stage Selection Step.

Local alignment is alignment which is performed on a partial sequence and in which it is possible to locally check a portion with high complementarity.

However, in the present invention, the local alignment is different from local alignment usually performed on a base sequence, and is designed such that partial sequences to be subjected to comparison include the 3′ terminals of both base sequences by performing local alignment under the condition that the “partial sequences to be subjected to comparison include the 3′ terminals of the base sequences”. Furthermore, in the present invention, an embodiment is preferable in which partial sequences to be subjected to comparison include the 3′ terminals of both base sequences by performing local alignment under the condition that the “partial sequences to be subjected to comparison include the 3′ terminals of the base sequences”, that is, the condition that “only alignments in which a partial sequence to be subjected to comparison begins at the 3′ terminal of one sequence and ends at the 3′ terminal of the other sequence”.

Local alignment may be performed by inserting a gap. The gap means insertion and/or deletion (indel) of a base.

In addition, in the local alignment, a case where bases are complementary to each other between base sequence pairs is regarded as a match and a case where bases are not complementary to each other therebetween is regarded as a mismatch.

Local alignment is performed such that scores for each of the match, the mismatch, and the indel are given and the total score (local alignment score) becomes a maximum. The scores to be given to each of the match, the mismatch, and the indel may be appropriately set. For example, scores to be given to each of the match, the mismatch, and the indel may be set as shown in Table 1. “−” in Table 1 represents a gap (insertion and/or deletion (indel)).

TABLE 1

| | A | T | G | C | — |
|---|---|---|---|---|---|
| A | −1 | +1 | −1 | −1 | −3 |
| T | +1 | −1 | −1 | −1 | −3 |
| G | −1 | −1 | −1 | +1 | −3 |
| C | −1 | −1 | +1 | −1 | −3 |
| — | −3 | −3 | −3 | −3 | |

“—”: Gap

For example, it is considered that local alignment is performed on base sequences of SEQ ID No: 1 and SEQ ID No: 2 shown in Table 2. Here, the scores to be given to each of the match, the mismatch, and the gap are as shown in Table 1.

TABLE 2
Base sequence (5′ → 3′)

| | |
|---|---|
| SEQ ID No: 1: | CGCTCTTCCGATCTCTGCTTCGATGCGGACCTTCTGG |
| SEQ ID No: 2: | CGCTCTTCCGATCTGACTCTCCCACATCCGGCTATGG |

From the base sequences of SEQ ID No: 1 and SEQ ID No: 2, a dot matrix shown in Table 3 is generated. Specifically, the base sequence of SEQ ID No: 1 is arranged from the left to the right in an orientation of 5′ to 3′ and the base sequence of SEQ ID No: 2 is arranged from the bottom to the top in an orientation of 5′ to 3′. “•” is filled in a grid of which bases are complementary to each other, and a dot matrix shown in Table 3 is obtained.

From the dot matrix shown in Table 3, alignment (pairwise alignment) of partial sequences shown in Table 4 is obtained (refer to a thick line portion of Table 3).

TABLE 4
Base sequence

| | |
|---|---|
| Partial | 5′• C T T C G A T G C G G A C C T T C T G G •3′ |
| sequence from | |
| SEQ ID No: 1: | |
| | \| : : : : : : * \| \| \| \| : : \| : : : : \| |
| Partial | 3′• G G T A T C G - G C C T A C A C C C T C •5′ |
| sequence from | |
| SEQ ID No: 2: | |

“|” = Match; “:” = Mismatch; “*” = Gap (indel)

Due to match (+1)×7, mismatch (−1)×12, and gap (−3)×1 from Table 4, the local alignment score regarding the local alignment is “−8”.

The alignment (pairwise alignment) can be obtained not only through the dot matrix method exemplified herein, but also through a dynamic programming method, a word method, or various other methods.

(d) First Stage Selection Step

(d) First Stage Selection Step is shown in the block diagram of FIG. 1 as “(n-th) STEP OF FIRST STAGE SELECTION”.

The first stage selection step is a step of performing first stage selection of the base sequence of the primer candidate for amplifying the target locus based on the local alignment score obtained in (c) Local Alignment Step.

A threshold value (first threshold value) of the local alignment score is predetermined.

In a case where a local alignment score of a pair of two base sequences is less than the first threshold value, it is determined that the pair of these two base sequences has low primer dimer formability, and the following step is performed. In contrast, in a case where a local alignment score of a pair of two base sequences is greater than or equal to the first threshold value, it is determined that the pair of these two base sequences has high primer dimer formability, and the following step is not performed on the pair.

The first threshold value is not particularly limited and can be appropriately set. For example, the first threshold value may be set using a PCR condition such as the amount of genomic DNA which becomes a template for a polymerase chain reaction.

Here, in the example in which (c) Local Alignment Step is shown, a case where the first threshold value is set to “3” is considered.

In the above-described example, the local alignment score is “−8” and is less than “3” which is the first threshold value. Therefore, it is possible to determine that the pair of the base sequences of SEQ ID No: 1 and SEQ ID No: 2 has low primer dimer formability.

The present step is performed on all of the pairs for which scores are calculated in (c) Local Alignment Step.

(e) Global Alignment Step

(e) Global Alignment Step is shown in the block diagram of FIG. 1 as “(n-th) GLOBAL ALIGNMENT STEP”.

The global alignment step is a step of obtaining a global alignment score by performing pairwise global alignment on a base sequence which has a predetermined sequence length and includes the 3′ terminal of the base sequence of the primer candidate for amplifying the target locus.

A combination of pairs of base sequences to be subjected to global alignment may be a combination selected while allowing overlapping, or may be a combination selected without allowing overlapping. However, in a case where formability of a primer dimer between primers of an identical base sequence has not yet been evaluated, the combination selected while allowing overlapping is preferable.

The total number of combinations is “ₘH₂=ₘ₊₁C₂=(m+1)!/2(m−1)!” in a case where the selection is performed while allowing overlapping, and is “ₘC₂=m(m−1)/2” in a case where the selection is performed without allowing overlapping, in which the number of base sequences which have been generated in (b) Primer Candidate Base Sequence Generation Step is set to be m.

In a case where both steps of (c) Local Alignment Step and (d) First Stage Selection Step which have been described above are performed first, the present step and (f) Second Stage Selection Step to be described below may be performed on primer candidates selected in (d) First Stage Selection Step.

Global alignment is an alignment which is performed on the entire sequence and in which it is possible to check complementarity of the entire sequence.

However, here, the “entire sequence” refers to the entirety of a base sequence which has a predetermined sequence length and includes the 3′ terminal of a base sequence of a primer candidate.

Global alignment may be performed by inserting a gap. The gap means insertion and/or deletion (indel) of a base.

In addition, in the global alignment, a case where bases are complementary to each other between base sequence pairs is regarded as a match and a case where bases are not complementary to each other therebetween is regarded as a mismatch.

Global alignment is performed such that scores for each of the match, the mismatch, and the indel are given and the total score (global alignment score) becomes a maximum. The scores to be given to each of the match, the mismatch, and the indel may be set appropriately. For example, scores to be given to each of the match, the mismatch, and the indel may be set as shown in Table 1. “−” in Table 1 represents a gap (insertion and/or deletion (indel)).

For example, it is considered that global alignment is performed on three bases (refer to portions with capital letters and correspond to the “base sequence which has a predetermined sequence length and includes the 3′ terminal”) at the 3′ terminal of each base sequence of SEQ ID No: 1 and SEQ ID No: 2 shown in Table 5. Here, the scores to be given to each of the match, the mismatch, and the gap are as shown in Table 1.

TABLE 5
Base sequence (5′ → 3′)

| | |
|---|---|
| SEQ ID | cgctcttccgatctctgcttcgatgcggaccttcTGG |
| No: 1: | |
| SEQ ID | cgctcttccgatctgactctcccacatccggctaTGG |
| No: 2: | |

Alignment (pairwise alignment) shown in Table 6 is obtained by performing global alignment on base sequences of the three bases (portion with capital letters) at the 3′ terminal of the base sequence of SEQ ID No: 1 and the three bases (portion with capital letters) at the 3′ terminal of SEQ ID No: 2 such that the score becomes a maximum.

TABLE 6

| | Base sequence | | | | |
|---|---|---|---|---|---|
| Three bases at 3′ terminal of SEQ ID No: 1: | 5′• | T | G | G | •3′ |
| | | : | : | : | |
| Three bases at 3′ terminal of SEQ ID No: 2: | 3′• | G | G | T | •5′ |

“:” = Mismatch

Due to match (+1)×0, mismatch (−1)×3, and gap (−3)×0 from Table 6, the global alignment score regarding the global alignment is “−3”.

The alignment (pairwise alignment) can be obtained through the dot matrix method a dynamic programming method, a word method, or various other methods.

(f) Second Stage Selection Step

(f) Second Stage Selection Step is shown in the block diagram of FIG. 1 as “(n-th) STEP OF SECOND STAGE SELECTION”.

The second stage selection step is a step of performing second stage selection of the base sequence of the primer candidate for amplifying the target locus based on the global alignment score obtained in (e) Global Alignment Step.

A threshold value (second threshold value) of the global alignment score is predetermined.

In a case where a global alignment score of a pair of two base sequences is less than the second threshold value, it is determined that the pair of these two base sequences has low primer dimer formability, and the following step is performed. In contrast, in a case where a global alignment score of a pair of two base sequences is greater than or equal to the second threshold value, it is determined that the pair of these two base sequences has high primer dimer formability, and the following step is not performed on the pair.

The second threshold value is not particularly limited and can be appropriately set. For example, the second threshold value may be set using a PCR condition such as the amount of genomic DNA which becomes a template for a polymerase chain reaction.

It is possible to set the global alignment score obtained by performing pairwise global alignment on a base sequence which has a predetermined number of bases and includes the 3′ terminal of a base sequence of each primer to be less than the second threshold value by setting a base sequence with several bases from the 3′ terminal of a primer as an identical base sequence.

Here, in the example in which (e) Global Alignment Step is shown, a case where the second threshold value is set to “3” is considered.

In the above-described example, the global alignment score is “−3” and is less than “3” which is the second threshold value. Therefore, it is possible to determine that the pair of the base sequences of SEQ ID No: 1 and SEQ ID No: 2 has low primer dimer formability.

The present step is performed on all of the pairs for which scores are calculated in (e) Global Alignment Step.

Both steps of (c) Local Alignment Step and (d) First Stage Selection Step may be performed before or after both steps of (e) Global Alignment Step and (f) Second Stage Selection Step, or may be performed in parallel with both steps of (e) Global Alignment Step and (f) Second Stage Selection Step.

In addition, in order to reduce the amount of calculation, it is preferable to perform both steps of (c) Local Alignment Step and (d) First Stage Selection Step in a combination which has passed (f) Second Stage Selection Step after first performing both steps of (e) Global Alignment Step and (f) Second Stage Selection Step. Particularly, as the number of target loci and the number of base sequences of primer candidates are increased, the effect of reducing the amount of calculation is increased, and it is possible to speed up the overall processing.

This is because the amount of calculation of a global alignment score is smaller than that of a local alignment score which is obtained by searching a partial sequence with high complementarity from the entire base sequence under the condition that the base sequence includes the 3′ terminal and it is possible to speed up the processing since global alignment is performed on a base sequence with a short length called a “predetermined sequence length” in (e) Global Alignment Step. It is known that the global alignment is faster than the local alignment in a case of alignment with respect to a sequence having an identical length in a well-known algorithm.

(Amplification Sequence Length-Checking Step)

As desired, an amplification sequence length-checking step may be performed.

The amplification sequence length-checking step is a step of calculating the distance between ends of base sequences of primer candidates for which it has been determined that formability of a primer dimer is low in (d) First Stage Selection Step and (f) Second Stage Selection Step, on genomic DNA or chromosomal DNA regarding pairs of the base sequences of the primer candidates, and determining whether the distance is within a predetermined range may be performed.

In a case where the distance between the ends of the base sequences is within the predetermined range, it is possible to determine that there is a high possibility that the pairs of the base sequences of the primer candidates can appropriately amplify a target locus. The distance between the ends of the base sequences of the primer candidates is not particularly limited, and can be appropriately set in accordance with the PCR condition such as the type of enzyme (DNA polymerase). For example, the distance between the ends of the base sequences of the primer candidates can be set to be within various ranges such as a range of 100 to 200 bases (pair), a range of 120 to 180 bases (pair), a range of 140 to 180 bases (pair) a range of 140 to 160 bases (pair), and a range of 160 to 180 bases (pair).

(g) Primer Employment Step

(g) Primer Employment Step is shown in the block diagram of FIG. 1 as “n-th PRIMER EMPLOYMENT STEP”.

The primer employment step is a step of employing a base sequence of a base sequence of a primer candidate which has been selected in both of (d) First Stage Selection Step and (f) Second Stage Selection Step, as a base sequence of a primer for amplifying the above-described target locus.

That is, in the present step, a base sequence of a primer candidate, in which a local alignment score obtained by performing pairwise local alignment on a base sequence of each primer candidate under a condition that a partial sequence to be subjected to comparison includes the 3′ terminal of the base sequence is less than the first threshold value, and a global alignment score obtained by performing pairwise global alignment on a base sequence which has a predetermined number of bases and includes the 3′ terminal of the base sequence of each primer candidate is less than the second threshold value, is employed as a base sequence of a primer for amplifying a target locus.

For example, it is considered that base sequences of SEQ ID No: 1 and SEQ ID No: 2 shown in Table 7 are employed as base sequences of primers for amplifying a target locus.

TABLE 7
Base sequence (5′ → 3′)

| | |
|---|---|
| SEQ ID | CGCTCTTCCGATCTCTGCTTCGATGCGGACCTTCTGG |
| No: 1: | |
| SEQ ID | CGCTCTTCCGATCTGACTCTCCCACATCCGGCTATGG |
| No: 2: | |

As already described, the local alignment score is “−8” and is less than “3” which is the first threshold value. Moreover, the global alignment score is “−3” and is less than “3” which is the second threshold value.

Accordingly, it is possible to employ the base sequence of the primer candidate represented by SEQ ID No: 1 and the base sequence of primer candidate represented by SEQ ID No: 2 as base sequences of primers for amplifying a target locus.

A second embodiment of the method for designing primer sets in the present invention includes the following steps:

(aₙ) an n-th target locus selection step of selecting an n-th target locus, in which primer sets used in multiplex PCR are designed, from a plurality of loci;

(bₙ) an n-th primer candidate base sequence generation step of generating at least one base sequence of a primer candidate for amplifying the n-th target locus regarding each of a forward-side primer and a reverse-side primer based on a base sequence in a vicinity region of the n-th target locus on the chromosomes;

(cₙ) an n-th local alignment step of obtaining a local alignment score by performing pairwise local alignment on the base sequence of the primer candidate for amplifying the n-th target locus under a condition that a partial sequence to be subjected to comparison includes the 3′ terminal of the base sequence of the primer candidate for amplifying the n-th target locus;

(dₙ) an n-th step of first stage selection of performing n-th stage selection of the base sequence of the primer candidate for amplifying the n-th target locus based on the local alignment score obtained in the n-th local alignment step;

(eₙ) a n-th global alignment step of obtaining a global alignment score by performing pairwise global alignment on a base sequence which has a predetermined sequence length and includes the 3′ terminal of the base sequence of the primer candidate for amplifying the n-th target locus;

(fₙ) an n-th step of second stage selection of performing second stage selection of the base sequence of the primer candidate for amplifying the n-th target locus based on the global alignment score obtained in the n-th global alignment step; and

(gₙ) an n-th primer employment step of employing the base sequence of the primer candidate which has been selected in both of the n-th step of first stage selection and the n-th step of second stage selection as a base sequence of a primer for amplifying the n-th target locus.

Here, n is an integer satisfying n≥1, and both steps of (cₙ) n-th Local Alignment Step and (dₙ) n-th Step of First Stage Selection may be performed before or after both steps of (eₙ) n-th Global Alignment Step and (fₙ) n-th Step of Second Stage Selection, or may be performed in parallel with both steps of (eₙ) n-th Global Alignment Step and (fₙ) n-th Step of Second Stage Selection.

In addition, in a case where the number N (N is an integer satisfying N≥2) of a plurality of loci is greater than n, the above-described n is replaced with n+1, and steps are repeated until primer sets are employed for all of the plurality of loci.

The steps in the case where n is replaced with n+1 are shown below.

(aₙ₊₁) An (n+1)th target locus selection step of selecting an (n+1)th target locus, which is different from the already selected target locus and in which primer sets used in multiplex PCR are designed, from the plurality of loci;

(bₙ₊₁) an (n+1)th primer candidate base sequence generation step of generating at least one base sequence of a primer candidate for amplifying the (n+1)th target locus regarding each of a forward-side primer and a reverse-side primer based on a base sequence in a vicinity region of the (n+1)th target locus on the chromosomes;

(cₙ₊₁) an (n+1)th local alignment step of obtaining a local alignment score by performing pairwise local alignment on the base sequence of the primer candidate for amplifying the (n+1)th target locus and the base sequence of the primer which has already been employed, under a condition that partial sequences to be subjected to comparison include the 3′ terminal of the base sequence of the primer candidate for amplifying the (n+1)th target locus and the 3′ terminal of the base sequence of the primer which has already been employed;

(dₙ₊₁) an (n+1)th step of first stage selection of performing first stage selection of the base sequence of the primer candidate for amplifying the (n+1)th target locus based on the local alignment score obtained in the (n+1)th local alignment step;

(eₙ₊₁) an (n+1)th global alignment step of obtaining a global alignment score by performing pairwise global alignment on base sequences which have a predetermined sequence length and include the 3′ terminal of the base sequence of the primer candidate for amplifying the (n+1)th target locus and the 3′ terminal of the base sequence of the primer which has already been employed;

(fₙ₊₁) an (n+1)th step of (n+1)th stage selection of performing (n+1)th stage selection of the base sequence of the primer candidate for amplifying the (n+1)th target locus based on the global alignment score obtained in the (n+1)th global alignment step; and (gₙ₊₁) an (n+1)th primer employment step of employing the base sequence of the primer candidate which has been selected in both of the (n+1)th step of first stage selection and the (n+1)th step of (n+1)th stage selection as a base sequence of a primer for amplifying the (n+1)th target locus.

Here, both steps of (cₙ₊₁) (n+1)th Local Alignment Step and (dₙ₊₁) (n+1)th Step of First Stage Selection may be performed before or after both steps of (eₙ₊₁) (n+1)th Global Alignment Step and (fₙ₊₁) (n+1)th Step of (n+1)th Stage Selection, or may be performed in parallel with both steps of (eₙ₊₁) (n+1)th Global Alignment Step and (fₙ₊₁) (n+1)th Step of (n+1)th Stage Selection.

Each step of the second embodiment of the method for designing primer sets in the present invention will be described in detail.

(aₙ) n-th Target Locus Selection Step

(aₙ) n-th Target Locus Selection Step is shown in the block diagram of FIG. 1 as “n-th TARGET LOCUS SELECTION STEP”.

(an) n-th Target Locus Selection Step is the same as “(a) Target Locus Selection Step” of the first embodiment except that an n-th target locus is selected.

However, in a case where n≥2, a locus different from the target locus selected up to an (n−1)th target locus selection step is selected.

In a case where n≥2, the selection of the n-th target locus can be simultaneously performed with the selection of an (n−1)th target locus, or can be performed after the selection of the (n−1)th target locus.

(bₙ) n-th Primer Candidate Base Sequence Generation Step

(bₙ) n-th Primer Candidate Base Sequence Generation Step is shown in the block diagram of FIG. 1 as “n-th PRIMER CANDIDATE BASE SEQUENCE GENERATION STEP”.

(bₙ) n-th Primer Candidate Base Sequence Generation Step is the same as “(b) Primer Candidate Base Sequence Generation Step” of the first embodiment of the method for designing primer sets of the present invention except that the base sequence of the primer candidate for amplifying the n-th target locus is generated.

(Specificity-Checking Step)

The specificity-checking step is the same as “Specificity-Checking Step” of the first embodiment of the method for designing primer sets of the present invention. The present step is an arbitrary step, and may be performed or may not be performed.

(cₙ) n-th Local Alignment Step

(cₙ) n-th Local Alignment Step is shown in the block diagram of FIG. 1 as “n-th LOCAL ALIGNMENT STEP”.

(cₙ) n-th Local Alignment Step is the same as “(c) Local Alignment Step” of the first embodiment of the method for designing primer sets of the present invention except that local alignment is performed on the base sequence of the primer candidate for amplifying the n-th target locus generated in (bₙ) n-th Primer Candidate Base Sequence Generation Step.

However, in a case where n≥2, local alignment is performed on the base sequence of the primer candidate for amplifying the n-th target locus generated in (bₙ) n-th Primer Candidate Base Sequence Generation Step and base sequences of primers which have already been employed. Here, all the base sequences of the primers which have already been employed are base sequences which have been employed as base sequences of primers for amplifying target loci from the first target locus to the (n−1)th target locus (the same applies hereinafter).

(dₙ) n-th Step of First Stage Selection

(dₙ) n-th Step of First Stage Selection is shown in the block diagram of FIG. 1 as “n-th STEP OF FIRST STAGE SELECTION”.

(dₙ) n-th Step of First Stage Selection is the same as “(d) First Stage Selection Step” of the first embodiment of the method for designing primer sets of the present invention except that the selection is performed on the base sequence of the primer candidate for amplifying the n-th target locus generated in (bₙ) n-th Primer Candidate Base Sequence Generation Step, based on the local alignment score obtained in (cₙ) n-th Local Alignment Step.

However, in a case where n≥2, selection is performed on the base sequence of the primer candidate for amplifying the n-th target locus generated in (bₙ) n-th Primer Candidate Base Sequence Generation Step and base sequences of primers which have already been employed.

(eₙ) n-th Global Alignment Step

(eₙ) n-th Global Alignment Step is shown in the block diagram of FIG. 1 as “n-th GLOBAL ALIGNMENT STEP”.

(eₙ) n-th Global Alignment Step is the same as “(e) Global Alignment Step” of the first embodiment of the method for designing primer sets of the present invention except that global alignment is performed on the base sequence of the primer candidate for amplifying the n-th target locus generated in (bₙ) n-th Primer Candidate Base Sequence Generation Step.

However, in a case where n≥2, global alignment is performed on the base sequence of the primer candidate for amplifying the n-th target locus generated in (bₙ) n-th Primer Candidate Base Sequence Generation Step and base sequences of primers which have already been employed.

(fₙ) n-th Step of Second Stage Selection

(fₙ) n-th Step of Second Stage Selection is shown in the block diagram of FIG. 1 as “n-th STEP OF SECOND STAGE SELECTION”.

(fₙ) n-th Step of Second Stage Selection is the same as “(f) Second Stage Selection Step” of the first embodiment of the method for designing primer sets of the present invention except that the selection is performed on the base sequence of the primer candidate for amplifying the n-th target locus generated in (bₙ) n-th Primer Candidate Base Sequence Generation Step, based on the global alignment score obtained in (eₙ) n-th Global Alignment Step.

However, in a case where n≥2, selection is performed on the base sequence of the primer candidate for amplifying the n-th target locus generated in (bₙ) n-th Primer Candidate Base Sequence Generation Step and base sequences of primers which have already been employed.

Similarly to the first embodiment of the method for designing primer sets of the present invention, both steps of (cₙ) n-th Local Alignment Step and (dₙ) n-th Step of First Stage Selection may be performed before or after both steps of (eₙ) n-th Global Alignment Step and (fₙ) n-th Step of Second Stage Selection, or may be performed in parallel with both steps of (eₙ) n-th Global Alignment Step and (fₙ) n-th Step of Second Stage Selection.

In addition, in order to reduce the amount of calculation, it is preferable to perform both steps of (cₙ) n-th Local Alignment Step and (dₙ) n-th Step of First Stage Selection in a combination which has passed (fₙ) n-th Step of Second Stage Selection after performing both steps of (eₙ) n-th Global Alignment Step and (fₙ) n-th Step of Second Stage Selection” first. Particularly, as the number of target loci and the number of base sequences of primer candidates are increased, the effect of reducing the amount of calculation is increased, and it is possible to speed up the overall processing.

(Amplification Sequence Length-Checking Step)

The specificity-checking step is the same as “Amplification Sequence Length-Checking Step” of the first embodiment of the method for designing primer sets of the present invention. The present step is an arbitrary step, and may be performed or may not be performed.

(gₙ) n-th Primer Employment Step

(gₙ) n-th Primer Employment Step is shown in the block diagram of FIG. 1 as “n-th PRIMER EMPLOYMENT STEP”.

(gₙ) n-th Primer Employment Step is the same as “(g) Primer Employment Step” of the first embodiment of the method for designing primer sets of the present invention.

Hereinafter, the present invention will be described in more detail using an example, but is not limited to these examples.

### EXAMPLES

[Single Cell Isolation and Genomic DNA Extraction]

<Single Cell Isolation>

(Acquisition of Peripheral Blood Sample)

10.5 mg of sodium salts of ethylenediaminetetraacetic acid (EDTA) was added to a 7 mL blood collecting tube as an anticoagulant, and then, 7 mL of peripheral blood was obtained within the blood collecting tube as volunteer blood after obtaining informed consent from a pregnant woman volunteer. Thereafter, the blood was diluted using physiological salt solution.

(Concentration of Nucleated Red Blood Cell)

A liquid with a density of 1.070 (g/cm³) and a liquid with a density of 1.095 (g/cm³) were prepared using PERCOLL LIQUID (manufactured by GE Healthcare Bioscience), 2 mL of a liquid with a density of 1.095 g/mL was added to the bottom portion of a centrifuge tube, and the centrifuge tube was cooled in a refrigerator for 30 minutes at 4° C.

Thereafter, the centrifuge tube was taken out from the refrigerator and 2 mL of a liquid with a density of 1.070 (g/cm³) was made to slowly overlap the top of the liquid with a density of 1.095 (g/cm³) so as not to disturb the interface.

Then, 11 mL of diluent of blood which had been collected above was slowly added to the top of the medium with a density of 1.070 (g/cm³) in the centrifuge tube.

Thereafter, centrifugation was performed for 20 minutes at 2,000 rpm.

The centrifuge tube was taken out and fractions which had been deposited between the liquid with a density of 1.070 (g/cm³) and the liquid with a density of 1.095 (g/cm³) were collected using a pipette.

A droplet of the fractions of blood which have been collected in this manner was spotted at one end of a slide glass substrate 1 while holding the slide glass substrate 1 using one hand. A slide glass substrate 2 was held by the other hand and one end of the slide glass substrate 2 was brought into contact with the slide glass substrate 1 at an angle of 30°. The contact surface of the slide glass substrate 2 which was brought into contact with the fractions of blood was then spread into the space surrounded by the two sheets of slide glass due to a capillary phenomenon.

Next, the slide glass substrate 2 was made to be slid in a direction of a region opposite to the region of the slide glass substrate 1, on which blood was placed, while maintaining the angle, and the slide glass substrate 1 was uniformly coated with blood. After the completion of coating, the slide glass substrate 1 was sufficiently dried through air blowing for one or more hours. This glass substrate was immersed in a MAY-Grunwald staining liquid for 3 minutes and was washed by being immersed in a phosphoric acid buffer solution. Thereafter, the glass substrate was immersed in a GIEMSA staining liquid, which was diluted with a phosphoric acid buffer solution to make a concentration of 3%, for 10 minutes.

Thereafter, a plurality of stained glass substrates were prepared by being dried after being washed with pure water.

(Identification of Nucleated Red Blood Cell Using information on Shape of Cell)

In order to sort out nucleated red blood cell candidates from the cells with which the top of the slide glass substrate was coated, a measurement system of an optical microscope provided with an electric XY stage, an objective lens, and a CCD camera, a control unit provided with an XY stage control unit and a Z-direction control unit, and a control unit portion including an image input unit, an image processing unit, and an XY position recording unit were prepared. Blood cells which had been prepared as described above and with which the top of the slide glass substrate was coated were placed on the XY stage and scanning was performed by performing focusing on the slide glass. An image which was obtained using an optical microscope was taken and nucleated red blood cells which were objective cells were searched through image analysis.

In the image analysis, cells which satisfied the two following conditions were detected and the XY position was recorded.

0.25<N/C<1.0 (1)

0.65<N/L²<0.785 (2)

Here, “N” represents the area of a nuclear region of a cell on which image analysis is to be performed, “C” represents the area of cytoplasm of a cell on which image analysis is to be performed, and “L” represents the length of the major axis of a nucleus of a cell on which image analysis is to be performed. The length of the major axis of a nucleus of a cell is defined as a length of the major axis of an elliptical shape circumscribing a cell nucleus which has a complicated shape.

Nucleated red blood cells which satisfy Formulas (1) and (2) were selected from nucleated red blood cells existing on the slide glass substrate, and were regarded as nucleated red blood cell candidates of the next step.

(Sorting of Fetal Nucleated Red Blood Cell)

Analysis of spectral information was performed on the nucleated red blood cell candidates, which had been identified in the step of identifying nucleated red blood cells using information on the shape of cells, using a microspectrometer.

The nucleated red blood cell candidates on the slide glass substrate were specified, one cell among them was irradiated with monochromatic light in the vicinity of 415 nm, and the absorption coefficient of the cell was measured.

Next, three white blood cells of which the shapes of nuclei in the vicinity of the cell did not satisfy Formula (2) were selected from cells closest to the nucleated red blood cell candidates. The absorption coefficient of each white blood cell was calculated in the same manner, and an average absorption coefficient was calculated.

The absorption coefficients of remaining cells of the nucleated red blood cell candidates on the slide glass substrate were also measured similarly to the above, and an average value of the absorption coefficients of three white blood cells in the vicinity of each cell was calculated. Cells of which the ratio of the absorption coefficient of a nucleated red blood cell candidate to the average absorption coefficient of the white blood cells becomes greater than or equal to 1 were extracted from these results. As a result, 8 cells of which the ratio was clearly greater than or equal to 1 were detected.

(Cell Collection)

The 8 cells determined as described above were collected using a micromanipulator.

<Genomic DNA Extraction>

Cytolysis was performed on the collected single cells using a Single Cell WGA kit (manufactured by New England Biolabs). That is, each of the single cells was mixed in 5 μL of Cell extraction buffer, 4.8 μL of Extraction Enzyme Dilution Buffer and 0.2 μL of Cell Extraction Enzyme were mixed with the mixture to make the total amount of the solution be 10 μL, the solution was incubated for 10 minutes at 75° C., and then, the solution was further incubated for 4 minutes at 95° C., in accordance with the description of “Sample Preparation Methods” and “Pre-Amplification Protocol” in an instruction attached to the kit.

Accordingly, genomic DNA was prepared.

### Example 1

<Selection of Locus to be Amplified through Multiplex PCR>

Chromosomes of interest are chromosome 13, chromosome 18, and chromosome 21. Regarding the number of loci to be amplified through multiplex PCR, the number of loci on chromosome 13 was set to 181, the number of loci on chromosome 18 was set to 178, the number of loci on chromosome 21 was set to 188, the number of loci on an X chromosome was set to 51, and the number of loci on a Y chromosome was set to 49 so that the total number of loci became 647. The selected loci are shown in Tables 8 to 12.

TABLE 8
Loci (coordinate) on chromosome 13

chr13: 19751127
chr13: 19751657
chr13: 20763113
chr13: 20763380
chr13: 21006355
chr13: 21205192
chr13: 21553872
chr13: 21555696
chr13: 21620085
chr13: 22255230
chr13: 23894812
chr13: 23898509
chr13: 23904976
chr13: 23913017
chr13: 24243004
chr13: 24432997
chr13: 24797383
chr13: 24797913
chr13: 24823699
chr13: 24860985
chr13: 24895805
chr13: 25009099
chr13: 25029218
chr13: 25265103
chr13: 25266932
chr13: 25280464
chr13: 25281181
chr13: 25453420
chr13: 25671755
chr13: 25743748
chr13: 26043182
chr13: 26144896
chr13: 26620924
chr13: 27256807
chr13: 27333412
chr13: 27845643
chr13: 28367956
chr13: 28537317
chr13: 28609723
chr13: 28636084
chr13: 28893642
chr13: 29599322
chr13: 29599723
chr13: 29600415
chr13: 29608252
chr13: 29855847
chr13: 30097543
chr13: 31318222
chr13: 31338174
chr13: 31711623
chr13: 32360511
chr13: 32785086
chr13: 33628138
chr13: 33635835
chr13: 33684151
chr13: 33704065
chr13: 35517239
chr13: 36348767
chr13: 36382373
chr13: 36385031
chr13: 36686138
chr13: 36801415
chr13: 36886469
chr13: 37679333
chr13: 38266328
chr13: 38924062
chr13: 39262057
chr13: 39262435
chr13: 39263023
chr13: 39263714
chr13: 39263961
chr13: 39265511
chr13: 39266138
chr13: 39266565
chr13: 39424253
chr13: 39438560
chr13: 39454452
chr13: 39587572
chr13: 41134003
chr13: 41134396
chr13: 41323397
chr13: 41379272
chr13: 41508049
chr13: 41515368
chr13: 41533052
chr13: 41808035
chr13: 41814520
chr13: 42872719
chr13: 43930140
chr13: 44457925
chr13: 45147990
chr13: 45149662
chr13: 46115731
chr13: 46124375
chr13: 46541673
chr13: 46638826
chr13: 46648094
chr13: 46946157
chr13: 49070345
chr13: 50126382
chr13: 50134099
chr13: 50204880
chr13: 50589718
chr13: 51918558
chr13: 52313195
chr13: 52348164
chr13: 52518383
chr13: 52971893
chr13: 53624380
chr13: 60240961
chr13: 61986918
chr13: 67800935
chr13: 67802095
chr13: 67802513
chr13: 76055820
chr13: 76395620
chr13: 76397731
chr13: 76423248
chr13: 76432051
chr13: 77570078
chr13: 77632470
chr13: 84455178
chr13: 86369981
chr13: 88328960
chr13: 95858978
chr13: 96205154
chr13: 96258288
chr13: 98828892
chr13: 98865546
chr13: 99030075
chr13: 99037076
chr13: 99100547
chr13: 99337039
chr13: 99356611
chr13: 99364165
chr13: 99447005
chr13: 99449468
chr13: 99449717
chr13: 99457431
chr13: 99907341
chr13: 99948097
chr13: 101736075
chr13: 102366825
chr13: 103389420
chr13: 103396716
chr13: 103397030
chr13: 103402099
chr13: 103528002
chr13: 108518444
chr13: 109496813
chr13: 110833702
chr13: 111077197
chr13: 111134858
chr13: 111154058
chr13: 111155773
chr13: 111287047
chr13: 111293915
chr13: 111932927
chr13: 111992247
chr13: 113053470
chr13: 113210444
chr13: 113473629
chr13: 113512574
chr13: 113532554
chr13: 113719264
chr13: 113728781
chr13: 113770068
chr13: 113801737
chr13: 113960845
chr13: 114088080
chr13: 114098849
chr13: 114150007
chr13: 114154419
chr13: 114307200
chr13: 114307693
chr13: 114309226
chr13: 114323997
chr13: 114325855
chr13: 114514771
chr13: 114762198
chr13: 115090193

TABLE 9
Loci (coordinate) on chromosome 18

chr18: 346821
chr18: 2890768
chr18: 2892188
chr18: 2914310
chr18: 2921592
chr18: 2922149
chr18: 2940811
chr18: 3071878
chr18: 3075712
chr18: 3115877
chr18: 3129399
chr18: 3151695
chr18: 3188976
chr18: 3457539
chr18: 3702419
chr18: 5416160
chr18: 5551142
chr18: 5956238
chr18: 6943264
chr18: 6965340
chr18: 6973197
chr18: 6977844
chr18: 6983194
chr18: 6986227
chr18: 6999665
chr18: 7888198
chr18: 7955213
chr18: 8379296
chr18: 8387065
chr18: 8783835
chr18: 8790019
chr18: 8796223
chr18: 8798185
chr18: 8803625
chr18: 8806945
chr18: 10485680
chr18: 10699017
chr18: 10699888
chr18: 10705744
chr18: 10706794
chr18: 10714830
chr18: 10726909
chr18: 10763007
chr18: 10800448
chr18: 11071481
chr18: 11884567
chr18: 12292025
chr18: 12971179
chr18: 13056333
chr18: 13059173
chr18: 19153494
chr18: 20951443
chr18: 21100240
chr18: 21122781
chr18: 21136274
chr18: 21140432
chr18: 21229102
chr18: 21390826
chr18: 21413869
chr18: 21441717
chr18: 21481203
chr18: 21485200
chr18: 21489153
chr18: 21496554
chr18: 22056766
chr18: 22804549
chr18: 22805428
chr18: 22807059
chr18: 23866349
chr18: 24126875
chr18: 24497240
chr18: 25543387
chr18: 28649042
chr18: 28934681
chr18: 28956904
chr18: 29122799
chr18: 29164577
chr18: 29178549
chr18: 29784171
chr18: 29848436
chr18: 29855491
chr18: 29867174
chr18: 29867688
chr18: 32156889
chr18: 32919934
chr18: 33552862
chr18: 33750046
chr18: 33779855
chr18: 33831189
chr18: 34162836
chr18: 34273228
chr18: 34311363
chr18: 34324091
chr18: 34378445
chr18: 34850846
chr18: 39647381
chr18: 42529996
chr18: 42530796
chr18: 42531834
chr18: 42532606
chr18: 42533130
chr18: 43206985
chr18: 43219877
chr18: 43258936
chr18: 43432600
chr18: 43490602
chr18: 43491853
chr18: 43492274
chr18: 43495660
chr18: 43496165
chr18: 44087565
chr18: 44098220
chr18: 44104697
chr18: 44114380
chr18: 44149474
chr18: 44470706
chr18: 44560429
chr18: 44561619
chr18: 44589450
chr18: 44626630
chr18: 44627245
chr18: 47017820
chr18: 47369758
chr18: 47414638
chr18: 47489410
chr18: 47500836
chr18: 47511113
chr18: 47563299
chr18: 47751130
chr18: 47777937
chr18: 47800179
chr18: 47803354
chr18: 48190412
chr18: 48327815
chr18: 48703073
chr18: 54814730
chr18: 55020359
chr18: 55143766
chr18: 55247336
chr18: 55317591
chr18: 55998093
chr18: 56067804
chr18: 56137685
chr18: 56184191
chr18: 56202982
chr18: 56246845
chr18: 59195354
chr18: 60027241
chr18: 60053990
chr18: 60241732
chr18: 60812027
chr18: 61154206
chr18: 61233861
chr18: 61264298
chr18: 61379825
chr18: 61390316
chr18: 61650896
chr18: 65181049
chr18: 66504093
chr18: 71816278
chr18: 72103918
chr18: 72109211
chr18: 72593032
chr18: 72897316
chr18: 72998703
chr18: 72999000
chr18: 72999819
chr18: 74153252
chr18: 74274762
chr18: 74639368
chr18: 74671768
chr18: 74680259
chr18: 74830377
chr18: 74980601
chr18: 77227476
chr18: 77287531
chr18: 77805778
chr18: 77894844

TABLE 10
Loci (coordinate) on chromosome 21

chr21: 15882622
chr21: 16340289
chr21: 19713821
chr21: 19756050
chr21: 27284122
chr21: 27372388
chr21: 27394285
chr21: 27451555
chr21: 27852641
chr21: 28122730
chr21: 28212760
chr21: 28214910
chr21: 28305212
chr21: 30316850
chr21: 30342933
chr21: 30365162
chr21: 30408670
chr21: 30464866
chr21: 31587677
chr21: 31691792
chr21: 31692173
chr21: 31709691
chr21: 31812772
chr21: 31866472
chr21: 31874211
chr21: 32201822
chr21: 32253513
chr21: 32638549
chr21: 32853499
chr21: 33068525
chr21: 33340639
chr21: 33346936
chr21: 33371123
chr21: 33678976
chr21: 33691710
chr21: 33694120
chr21: 33719343
chr21: 33755763
chr21: 33867447
chr21: 33881034
chr21: 33921989
chr21: 33962803
chr21: 33976489
chr21: 34072139
chr21: 34619143
chr21: 34634991
chr21: 34749253
chr21: 34787312
chr21: 34975846
chr21: 35237608
chr21: 35260481
chr21: 35467645
chr21: 35721596
chr21: 35757862
chr21: 37233883
chr21: 37444822
chr21: 37444973
chr21: 37518706
chr21: 37610969
chr21: 37612139
chr21: 37617575
chr21: 37618190
chr21: 37752637
chr21: 37833407
chr21: 37904343
chr21: 37975323
chr21: 38117505
chr21: 38285420
chr21: 38309459
chr21: 38439597
chr21: 38568009
chr21: 38600557
chr21: 39671476
chr21: 39930986
chr21: 40190443
chr21: 40191431
chr21: 40338115
chr21: 40777873
chr21: 40873485
chr21: 41032740
chr21: 41295995
chr21: 41361796
chr21: 41559182
chr21: 41621714
chr21: 41652905
chr21: 41684090
chr21: 42812891
chr21: 42813714
chr21: 42817937
chr21: 43032387
chr21: 43161103
chr21: 43162206
chr21: 43169357
chr21: 43221797
chr21: 43242946
chr21: 43255625
chr21: 43325863
chr21: 43327117
chr21: 43412853
chr21: 43424851
chr21: 43436743
chr21: 43509956
chr21: 43510437
chr21: 43514676
chr21: 43519032
chr21: 43520551
chr21: 43522349
chr21: 43523594
chr21: 43524018
chr21: 43546509
chr21: 43704683
chr21: 43705994
chr21: 43708041
chr21: 43764586
chr21: 43783418
chr21: 43792869
chr21: 43805637
chr21: 43807322
chr21: 43808526
chr21: 43846762
chr21: 43852232
chr21: 43853775
chr21: 43856895
chr21: 43864694
chr21: 43867288
chr21: 43873037
chr21: 43913135
chr21: 43954936
chr21: 44180443
chr21: 44189166
chr21: 44306874
chr21: 44323720
chr21: 44324365
chr21: 44473980
chr21: 45211298
chr21: 45217905
chr21: 45379986
chr21: 45542193
chr21: 45656774
chr21: 45713737
chr21: 45732116
chr21: 45738376
chr21: 45876620
chr21: 45987736
chr21: 46032094
chr21: 46057391
chr21: 46313442
chr21: 46320313
chr21: 46600355
chr21: 46612428
chr21: 46624583
chr21: 46900410
chr21: 46902703
chr21: 46924383
chr21: 47243535
chr21: 47296634
chr21: 47349899
chr21: 47351282
chr21: 47355726
chr21: 47361589
chr21: 47404302
chr21: 47544599
chr21: 47545482
chr21: 47614443
chr21: 47627429
chr21: 47632006
chr21: 47636469
chr21: 47639492
chr21: 47639800
chr21: 47641794
chr21: 47660086
chr21: 47662759
chr21: 47704896
chr21: 47775389
chr21: 47776780
chr21: 47782264
chr21: 47786494
chr21: 47811272
chr21: 47836206
chr21: 47851777
chr21: 47954017
chr21: 47954427
chr21: 47957354
chr21: 47966843
chr21: 47969793
chr21: 47975686
chr21: 47977678
chr21: 47985655

TABLE 11
Loci (coordinate) on X chromosome

chrX: 2779570
chrX: 2951434
chrX: 3241050
chrX: 6995417
chrX: 14027177
chrX: 16168467
chrX: 16627756
chrX: 17746244
chrX: 19375782
chrX: 22291606
chrX: 26157220
chrX: 27839572
chrX: 30261002
chrX: 39932907
chrX: 43603391
chrX: 46472826
chrX: 48418126
chrX: 49061742
chrX: 50130544
chrX: 50659280
chrX: 53577887
chrX: 69250308
chrX: 69261818
chrX: 69478749
chrX: 69572490
chrX: 69749852
chrX: 69890301
chrX: 70146475
chrX: 84363140
chrX: 96139406
chrX: 108708552
chrX: 112024157
chrX: 117527020
chrX: 118219347
chrX: 122537277
chrX: 125955199
chrX: 134483151
chrX: 134991078
chrX: 135593337
chrX: 136112707
chrX: 141290865
chrX: 151129822
chrX: 152226542
chrX: 153048403
chrX: 153049739
chrX: 153051907
chrX: 153070999
chrX: 153132261
chrX: 153171993
chrX: 153219665
chrX: 153633359

TABLE 12
Loci (coordinate) on Y chromosome

chrY: 2710802
chrY: 2837564
chrY: 6740540
chrY: 6946990
chrY: 7229757
chrY: 7262370
chrY: 7526916
chrY: 7679062
chrY: 7961690
chrY: 8113984
chrY: 8396638
chrY: 8624945
chrY: 8632095
chrY: 8892984
chrY: 9114110
chrY: 9878799
chrY: 10038191
chrY: 13209049
chrY: 13864997
chrY: 14082469
chrY: 14655058
chrY: 14705721
chrY: 14756402
chrY: 14944834
chrY: 15053566
chrY: 15238478
chrY: 15716813
chrY: 15872674
chrY: 16021217
chrY: 16325632
chrY: 16963396
chrY: 17111947
chrY: 17115626
chrY: 17358415
chrY: 17753399
chrY: 17833136
chrY: 18811624
chrY: 18956584
chrY: 21156181
chrY: 21906335
chrY: 21924529
chrY: 22902924
chrY: 22921056
chrY: 23232726
chrY: 23239880
chrY: 23297891
chrY: 23370399
chrY: 23428429
chrY: 24377754

<Design of Primer Set>

A primer set used in multiplex PCR was designed, selected, and employed according to the above-described method for designing primer sets.

The primer names, the base sequences, and the SEQ ID Nos of 20 pairs of target loci among each of the target loci on chromosome 13, chromosome 18, chromosome 21, an X chromosome, and a Y chromosome employed as primer sets for PCR amplification are shown in Tables 13 to 17.

In a case of designing the primer sets, the size of an amplification product was set to 140 bp to 180 bp, the Tm value was set to 60° C. to 70° C., and the length of a complementary portion of a primer was set to 20 mer.

Selection of primer sets was performed by calculating scores using the scoring system shown in Table 1 and setting all of the threshold values of a local alignment score and a global alignment score to “+3”.

TABLE 13
Primer set for chromosome 13

| | | SEQ |
|---|---|---|
| Primer | | ID |
| name | Base sequence (5′ → 3′) | No |
| chr13: | CGCTCTTCCGATCTCTGCTTCGATGCGGACCTTCTGG | 1 |
| 20763380: | | |
| Fwd | | |
| chr13: | CGCTCTTCCGATCTGACTCTCCCACATCCGGCTATGG | 2 |
| 20763380: | | |
| Rev | | |
| chr13: | CGCTCTTCCGATCTCTGTTTCCCCGACCATAAGCTTG | 3 |
| 21205192: | | |
| Fwd | | |
| chr13: | CGCTCTTCCGATCTGACATACAGGGCTGAGAGATTGG | 4 |
| 21205192: | | |
| Rev | | |
| chr13: | CGCTCTTCCGATCTCTGTGATAAGGTCCGAACTTTGG | 5 |
| 21620085: | | |
| Fwd | | |
| chr13: | CGCTCTTCCGATCTGACGCGACTGCAAGAGATTCGTG | 6 |
| 21620085: | | |
| Rev | | |
| chr13: | CGCTCTTCCGATCTCTGATTTGCTGCTGACCAGGGTG | 7 |
| 23898509: | | |
| Fwd | | |
| chr13: | CGCTCTTCCGATCTGACAGGTACAGCTTCCCATCTGG | 8 |
| 23898509: | | |
| Rev | | |
| chr13: | CGCTCTTCCGATCTCTGCCGTGTGTGAGATTCTCGTG | 9 |
| 24797913: | | |
| Fwd | | |
| chr13: | CGCTCTTCCGATCTGACACTGCTCAGGGTCCTCTGTG | 10 |
| 24797913: | | |
| Rev | | |
| chr13: | CGCTCTTCCGATCTCTGGTAAAGCCTCCAGGATGTTG | 11 |
| 25009099: | | |
| Fwd | | |
| chr13: | CGCTCTTCCGATCTGACCTGGCACTTGTGCTGACTGG | 12 |
| 25009099: | | |
| Rev | | |
| chr13: | CGCTCTTCCGATCTCTGCCAAAGCGCACTCACCTGTG | 13 |
| 25029218: | | |
| Fwd | | |
| chr13: | CGCTCTTCCGATCTGACTAGCCAGTGAGAGCGAAGTG | 14 |
| 25029218: | | |
| Rev | | |
| chr13: | CGCTCTTCCGATCTCTGGGCCTAGAGGACGATGCTTG | 15 |
| 25265103: | | |
| Fwd | | |
| chr13: | CGCTCTTCCGATCTGACTGTTGATAACCATGCCGGTG | 16 |
| 25265103: | | |
| Rev | | |
| chr13: | CGCTCTTCCGATCTCTGTGCTGGACAGTGACTCATGG | 17 |
| 25266932: | | |
| Fwd | | |
| chr13: | CGCTCTTCCGATCTGACCATTTTCCTGTCCTGGCTTG | 18 |
| 25266932: | | |
| Rev | | |
| chr13: | CGCTCTTCCGATCTCTGATCCAGTTCATATGCCGTTG | 19 |
| 25453420: | | |
| Fwd | | |
| chr13: | CGCTCTTCCGATCTGACGCGTTGCTGTCATTCCTTTG | 20 |
| 25453420: | | |
| Rev | | |

In addition, regarding the primer consisting of a base sequence of SEQ ID No: 1 and the primer consisting of a base sequence of SEQ ID No: 2, local alignment performed under the condition of inclusion of the 3′ terminal according to the method for designing primer sets of the present invention, a local alignment score, global alignment performed on three bases of the 3′ terminal of the primers according to the method for designing primer sets of the present invention, and a global alignment score are shown in FIG. 2.

As shown in FIG. 2, the base sequence of SEQ ID No: 1 and the base sequence of SEQ ID No: 2 had a local alignment score of “−8” and global alignment score of “−3”, both of which were less than the set threshold value.

TABLE 14
Primer set for chromosome 18

| | | SEQ |
|---|---|---|
| Primer | | ID |
| name | Base sequence (5′ → 3′) | No |
| chr18: | CGCTCTTCCGATCTCTGAGGTTCTGCTCGTTGGCTTG | 21 |
| 346821: | | |
| Fwd | | |
| chr18: | CGCTCTTCCGATCTGACCCAAGAAGGACACGGATTGG | 22 |
| 346821: | | |
| Rev | | |
| chr18: | CGCTCTTCCGATCTCTGAGCTTCCCGGGAAATTAGTG | 23 |
| 3075712: | | |
| Fwd | | |
| chr18: | CGCTCTTCCGATCTGACGAATTTTAATCGCCCCTGTG | 24 |
| 3075712: | | |
| Rev | | |
| chr18: | CGCTCTTCCGATCTCTGGGACTGCTTAGATGCCGTGG | 25 |
| 3188976: | | |
| Fwd | | |
| chr18: | CGCTCTTCCGATCTGACAGTCAAAAGCATGTCAGTGG | 26 |
| 3188976: | | |
| Rev | | |
| chr18: | CGCTCTTCCGATCTCTGCTCTGTGGAGTCCGTGATGG | 27 |
| 3457539: | | |
| Fwd | | |
| chr18: | CGCTCTTCCGATCTGACATGCAGTCACAGTGGTATGG | 28 |
| 3457539: | | |
| Rev | | |
| chr18: | CGCTCTTCCGATCTCTGGGTAATGCTGCAAGCTCTGG | 29 |
| 5416160: | | |
| Fwd | | |
| chr18: | CGCTCTTCCGATCTGACCCTAGGGGATCAAGATGTGG | 30 |
| 5416160: | | |
| Rev | | |
| chr18: | CGCTCTTCCGATCTCTGATTCATCCCCTGACTTCTTG | 31 |
| 5956238: | | |
| Fwd | | |
| chr18: | CGCTCTTCCGATCTGACTATTTGCAGCAGATCGATGG | 32 |
| 5956238: | | |
| Rev | | |
| chr18: | CGCTCTTCCGATCTCTGCACCAACATAAATGGGATTG | 33 |
| 6943264: | | |
| Fwd | | |
| chr18: | CGCTCTTCCGATCTGACGCCACTGTGCTCTGTGATGG | 34 |
| 6943264: | | |
| Rev | | |
| chr18: | CGCTCTTCCGATCTCTGAGTCCTGTGAGCATCTCTGG | 35 |
| 6983194: | | |
| Fwd | | |
| chr18: | CGCTCTTCCGATCTGACTGAGTGAATTGGCAAGTTTG | 36 |
| 6983194: | | |
| Rev | | |
| chr18: | CGCTCTTCCGATCTCTGGCAGTTTGCAGGGACTCTTG | 37 |
| 8796223: | | |
| Fwd | | |
| chr18: | CGCTCTTCCGATCTGACCACTCCAGGTTCGCTCATGG | 38 |
| 8796223: | | |
| Rev | | |
| chr18: | CGCTCTTCCGATCTCTGCTCCTGCCTGTTTCAGGGTG | 39 |
| 8798185: | | |
| Fwd | | |
| chr18: | CGCTCTTCCGATCTGACAATCTGCACCCGGGAGTGTG | 40 |
| 8798185: | | |
| Rev | | |

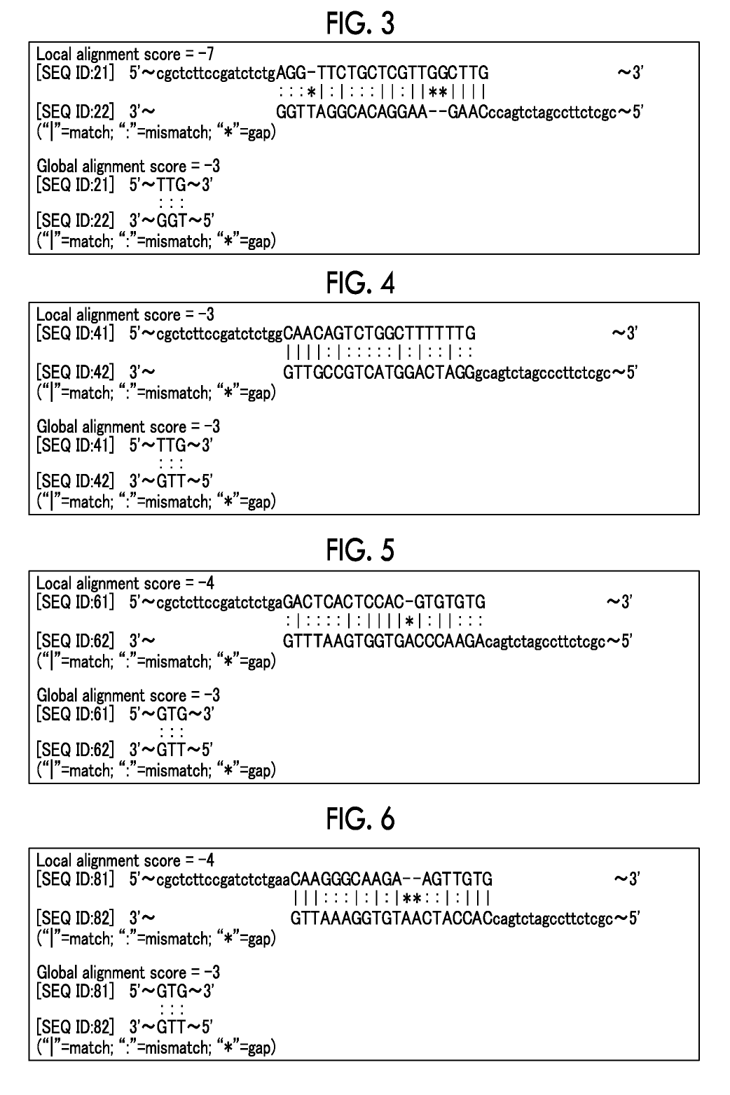

In addition, regarding the primer consisting of a base sequence of SEQ ID No: 21 and the primer consisting of a base sequence of SEQ ID No: 22, local alignment performed under the condition of inclusion of the 3′ terminal according to the method for designing primer sets of the present invention, a local alignment score, global alignment performed on three bases of the 3′ terminal of the primers according to the method for designing primer sets of the present invention, and a global alignment score are shown in FIG. 3.

As shown in FIG. 3, the base sequence of SEQ ID No: 21 and the base sequence of SEQ ID No: 22 had a local alignment score of “−7” and global alignment score of “−3”, both of which were less than the set threshold value.

TABLE 15
Primer set for chromosome 21

| | | SEQ |
|---|---|---|
| Primer | | ID |
| name | Base sequence (5′ → 3′) | No |
| chr21: | CGCTCTTCCGATCTCTGGCAACAGTCTGGCTTTTTTG | 41 |
| 16340289: | | |
| Fwd | | |
| chr21: | CGCTCTTCCGATCTGACGGGATCAGGTACTGCCGTTG | 42 |
| 16340289: | | |
| Rev | | |
| chr21: | CGCTCTTCCGATCTCTGCAAGGTGCTACATGTGCTGG | 43 |
| 28212760: | | |
| Fwd | | |
| chr21: | CGCTCTTCCGATCTGACCCTTTGCAGGGGAATGTTTG | 44 |
| 28212760: | | |
| Rev | | |
| chr21: | CGCTCTTCCGATCTCTGGAGCAGCGTACCATTGGGTG | 45 |
| 28305212: | | |
| Fwd | | |
| chr21: | CGCTCTTCCGATCTGACCAGTGTTCTCGCTCATGTGG | 46 |
| 28305212: | | |
| Rev | | |
| chr21: | CGCTCTTCCGATCTCTGTGTCTCCCCCTTTTTAGTTG | 47 |
| 30408670: | | |
| Fwd | | |
| chr21: | CGCTCTTCCGATCTGACTTTACCTGGCTTTGGAGTTG | 48 |
| 30408670: | | |
| Rev | | |
| chr21: | CGCTCTTCCGATCTCTGGTCAGCAAGTTGGCTACTGG | 49 |
| 30464866: | | |
| Fwd | | |
| chr21: | CGCTCTTCCGATCTGACAGCCTTAGGCTCCCATGGTG | 50 |
| 30464866: | | |
| Rev | | |
| chr21: | CGCTCTTCCGATCTCTGTGCTGAGTGCTTTCTGATTG | 51 |
| 31709691: | | |
| Fwd | | |
| chr21: | CGCTCTTCCGATCTGACTCACAGACGATAGCTGTGTG | 52 |
| 31709691: | | |
| Rev | | |
| chr21: | CGCTCTTCCGATCTCTGCTTCTCCTCCTGCTGTTTTG | 53 |
| 31812772: | | |
| Fwd | | |
| chr21: | CGCTCTTCCGATCTGACCCAAAGTGCAGGATGTCTGG | 54 |
| 31812772: | | |
| Rev | | |
| chr21: | CGCTCTTCCGATCTCTGGAGACTCCTCCCACTGGTTG | 55 |
| 32253513: | | |
| Fwd | | |
| chr21: | CGCTCTTCCGATCTGACAACCCTTGCCAGGTGACTTG | 56 |
| 32253513: | | |
| Rev | | |
| chr21: | CGCTCTTCCGATCTCTGTCTTCAGCAGCAGCTGGTGG | 57 |
| 32638549: | | |
| Fwd | | |
| chr21: | CGCTCTTCCGATCTGACCCAATTCCTTGGGTGACTTG | 58 |
| 32638549: | | |
| Rev | | |
| chr21: | CGCTCTTCCGATCTCTGGCGGAACCCATGTACCTGTG | 59 |
| 33694120: | | |
| Fwd | | |
| chr21: | CGCTCTTCCGATCTGACAAACAGAGCAGAAGTGGGTG | 60 |
| 33694120: | | |
| Rev | | |

In addition, regarding the primer consisting of a base sequence of SEQ ID No: 41 and the primer consisting of a base sequence of SEQ ID No: 42, local alignment performed under the condition of inclusion of the 3′ terminal according to the method for designing primer sets of the present invention, a local alignment score, global alignment performed on three bases of the 3′ terminal of the primers according to the method for designing primer sets of the present invention, and a global alignment score are shown in FIG. 4.

As shown in FIG. 4, the base sequence of SEQ ID No: 41 and the base sequence of SEQ ID No: 42 had a local alignment score of “−3” and global alignment score of “−3”, both of which were less than the set threshold value.

TABLE 16
Primer set for X chromosome

| | | SEQ |
|---|---|---|
| Primer | | ID |
| name | Base sequence (5′ → 3′) | No |
| chrX: | CGCTCTTCCGATCTCTGAGACTCACTCCACGTGTGTG | 61 |
| 2779570: | | |
| Fwd | | |
| chrX: | CGCTCTTCCGATCTGACAGAACCCAGTGGTGAATTTG | 62 |
| 2779570: | | |
| Rev | | |
| chrX: | CGCTCTTCCGATCTCTGCTTCCCCTTCTGTGGGTGTG | 63 |
| 2951434: | | |
| Fwd | | |
| chrX: | CGCTCTTCCGATCTGACAGGATAAAACAATGGGTTGG | 64 |
| 2951434: | | |
| Rev | | |
| chrX: | CGCTCTTCCGATCTCTGCTGTTGCCGTCTCTTCATGG | 65 |
| 3241050: | | |
| Fwd | | |
| chrX: | CGCTCTTCCGATCTGACACCTCTGGAGGAAGTTGTTG | 66 |
| 3241050: | | |
| Rev | | |
| chrX: | CGCTCTTCCGATCTCTGGAACTCCTTGTGGCGGCTTG | 67 |
| 6995417: | | |
| Fwd | | |
| chrX: | CGCTCTTCCGATCTGACCCTGCAAGAAGGTCTTATGG | 68 |
| 6995417: | | |
| Rev | | |
| chrX: | CGCTCTTCCGATCTCTGCTCCATGGCTTGGATCTTGG | 69 |
| 14027177: | | |
| Fwd | | |
| chrX: | CGCTCTTCCGATCTGACAGCGCCTGGACAGCTATGTG | 70 |
| 14027177: | | |
| Rev | | |
| chrX: | CGCTCTTCCGATCTCTGTACGCCAGGTGTCTCGCTTG | 71 |
| 16168467: | | |
| Fwd | | |
| chrX: | CGCTCTTCCGATCTGACTCCAGATAAAGGCGGCTTTG | 72 |
| 16168467: | | |
| Rev | | |
| chrX: | CGCTCTTCCGATCTCTGGACAGTTTGCAACCCTGTTG | 73 |
| 16627756: | | |
| Fwd | | |
| chrX: | CGCTCTTCCGATCTGACTCTGCTTTAATCGCATCGTG | 74 |
| 16627756: | | |
| Rev | | |
| chrX: | CGCTCTTCCGATCTCTGTGCAGGTCTGGAGGAAGTTG | 75 |
| 17746244: | | |
| Fwd | | |
| chrX: | CGCTCTTCCGATCTGACTACCAGGCACTTTGTCATGG | 76 |
| 17746244: | | |
| Rev | | |
| chrX: | CGCTCTTCCGATCTCTGTAATAAAGGGCCTGCGTTTG | 77 |
| 19375782: | | |
| Fwd | | |
| chrX: | CGCTCTTCCGATCTGACCACTCATACTGTGTCCGTGG | 78 |
| 19375782: | | |
| Rev | | |
| chrX: | CGCTCTTCCGATCTCTGAGTTACCAGCGCTTCGCTTG | 79 |
| 22291606: | | |
| Fwd | | |
| chrX: | CGCTCTTCCGATCTGACATGTTGCACAGACGGTAGTG | 80 |
| 22291606: | | |
| Rev | | |

In addition, regarding the primer consisting of a base sequence of SEQ Ill No: 61 and the primer consisting of a base sequence of SEQ ID No: 62, local alignment performed under the condition of inclusion of the 3′ terminal according to the method for designing primer sets of the present invention, a local alignment score, global alignment performed on three bases of the 3′ terminal of the primers according to the method for designing primer sets of the present invention, and a global alignment score are shown in FIG. 5.

As shown in FIG. 5, the base sequence of SEQ ID No: 61 and the base sequence of SEQ ID No: 62 had a local alignment score of “−4” and global alignment score of “−3”, both of which were less than the set threshold value.

TABLE 17
Primer set for Y chromosome

| | | SEQ |
|---|---|---|
| Primer | | ID |
| name | Base sequence (5′ → 3′) | No |
| chrY: | CGCTCTTCCGATCTCTGAACAAGGGCAAGAAGTTGTG | 81 |
| 2710802: | | |
| Fwd | | |
| chrY: | CGCTCTTCCGATCTGACCACCATCAATGTGGAAATTG | 82 |
| 2710802: | | |
| Rev | | |
| chrY: | CGCTCTTCCGATCTCTGCTTCAGCACAGATGTTTTGG | 83 |
| 6740540: | | |
| Fwd | | |
| chrY: | CGCTCTTCCGATCTGACTTTTGTTTGCCTGCCTTGTG | 84 |
| 6740540: | | |
| Rev | | |
| chrY: | CGCTCTTCCGATCTCTGTGTCATCAATAGTTGGCTGG | 85 |
| 7262370: | | |
| Fwd | | |
| chrY: | CGCTCTTCCGATCTGACAGTTCCCTTTTGTAGGGTGG | 86 |
| 7262370: | | |
| Rev | | |
| chrY: | CGCTCTTCCGATCTCTGTTGCTGTGTGAAGTCCTTGG | 87 |
| 8624945: | | |
| Fwd | | |
| chrY: | CGCTCTTCCGATCTGACAAGCGGACAGCTGTGTCTGG | 88 |
| 8624945: | | |
| Rev | | |
| chrY: | CGCTCTTCCGATCTCTGCCTGGGAGCTCGTGAGTTTG | 89 |
| 8632095: | | |
| Fwd | | |
| chrY: | CGCTCTTCCGATCTGACTCACGTCTGCCTAGATTTTG | 90 |
| 8632095: | | |
| Rev | | |
| chrY: | CGCTCTTCCGATCTCTGCAGAGTATCAGGCCTTCTGG | 91 |
| 14655058: | | |
| Fwd | | |
| chrY: | CGCTCTTCCGATCTGACGGCTTACCAGCTTGTAGTGG | 92 |
| 14655058: | | |
| Rev | | |
| chrY: | CGCTCTTCCGATCTCTGCCAACCATCACGAAAATTGG | 93 |
| 14756402: | | |
| Fwd | | |
| chrY: | CGCTCTTCCGATCTGACTCGTCTCGTACTGGAGATTG | 94 |
| 14756402: | | |
| Rev | | |
| chrY: | CGCTCTTCCGATCTCTGTGATACTCCAATTGTGGTGG | 95 |
| 14944834: | | |
| Fwd | | |
| chrY: | CGCTCTTCCGATCTGACCTGTGTTTTTCTTTGCGGTG | 96 |
| 14944834: | | |
| Rev | | |
| chrY: | CGCTCTTCCGATCTCTGCCCCAGTGGACAGAGTTTTG | 97 |
| 15872674: | | |
| Fwd | | |
| chrY: | CGCTCTTCCGATCTGACGGAGCCAATGCTGTGATGTG | 98 |
| 15872674: | | |
| Rev | | |
| chrY: | CGCTCTTCCGATCTCTGTGACATTGAAGGTAGCGTTG | 99 |
| 22902924: | | |
| Fwd | | |
| chrY: | CGCTCTTCCGATCTGACGAGAAATCGGAGTTCATTGG | 100 |
| 22902924: | | |
| Rev | | |

In addition, regarding the primer consisting of a base sequence of SEQ ID No: 81 and the primer consisting of a base sequence of SEQ ID No: 82, local alignment performed under the condition of inclusion of the 3′ terminal according to the method for designing primer sets of the present invention, a local alignment score, global alignment performed on three bases of the 3′ terminal of the primers according to the method for designing primer sets of the present invention, and a global alignment score are shown in FIG. 6.

As shown in FIG. 6, the base sequence of SEQ ID No: 81 and the base sequence of SEQ ID No: 82 had a local alignment score of “−4” and global alignment score of “−3”, both of which were less than the set threshold value.

<Singleplex PCR>

It was confirmed that primer sets employed by performing singleplex PCR through the following procedure could amplify target loci.

2 μL of genomic DNA (0.5 ng/μL) prepared from a large number of cells (including cells having a Y chromosome), 2 μL of a primer mix, 12.5 μL of a multiplex PCR mix 2 (manufactured by TAKARA BIO INC.), 0.125 μL of a multiplex PCR mix 1 (manufactured by TAKARA BIO INC.), and a proper amount of water were mixed with each other to prepare 25 μL of a final amount of a reaction solution.

The above-described primer mix is a mix obtained by mixing primers of primer sets such that the final concentration of the primers becomes 50 nM. The above-described multiplex PCR mix 1 and the above-described multiplex PCR mix 2 are reagents contained in MULTIPLEX PCR ASSAY KIT (manufactured by TAKARA BIO INC.).

After performing initial thermal denaturation for 60 seconds at 94° C. using each of the prepared reaction solutions, a thermal cycle of thermal denaturation performed for 30 seconds at 94° C., annealing performed for 90 seconds at 60° C., and an elongation reaction performed for 30 seconds at 72° C. was repeated 30 cycles to perform singleplex PCR.

A part of the reaction solution on which the singleplex PCR was performed was subjected to agarose gel electrophoresis to check whether or not amplification has been performed.

<Multiplex PCR>

(Amplification of Target Loci)

2 μL of extracted genomic DNA (0.5 ng/μL), 2 μL of a primer mix, 12.5 μL of a multiplex PCR mix 2 (manufactured by TAKARA BIO INC.), 0.125 μL of a multiplex PCR mix 1 (manufactured by TAKARA BIO INC.), and a proper amount of water were mixed with each other to prepare 25 μL of a final amount of a reaction solution.

The above-described primer mix is a mix obtained by mixing a primer set for amplifying loci at 181 positions on chromosome 13, a primer set for amplifying loci at 178 positions on chromosome 18, a primer set for amplifying loci at 188 positions on chromosome 21, a primer set for amplifying loci at 51 positions on an X chromosome, and a primer set for amplifying loci at 49 positions on a Y chromosome with each other such that the final concentration of the primers becomes 50 nM. In addition, the above-described multiplex PCR mix 1 and the above-described multiplex PCR mix 2 are reagents contained in MULTIPLEX PCR ASSAY KIT (manufactured by TAKARA BIO INC.).

After performing initial thermal denaturation for 60 seconds at 94° C. using each of the prepared reaction solution, a thermal cycle of thermal denaturation performed for 30 seconds at 94° C., annealing performed for 90 seconds at 60° C., and an elongation reaction performed for 30 seconds at 72° C. was repeated 35 cycles to perform multiplex PCR.

<DNA Sequencing>

(Purification of PCR Amplification Product)

PCR amplification products obtained through multiplex PCR were purified using a spin column (QIAquick PCR Purification Kit manufactured by QIAGEN). In addition, the PCR amplification products may be purified using magnetic beads (for example, AMPure manufactured by Beckman Coulter Inc.).

(Addition of Index Sequence and Sequence for Bonding Flow Cell)

Next, an index sequence for identifying a sample, and P5 and P7 sequences for bonding a flow cell were added to both terminals of the multiplex PCR amplification products in order to perform a sequencing reaction using Miseq (manufactured by Illumina, Inc.). 1.25 μM of each D501-F (SEQ ID No: 101), D701-R (SEQ ID No: 102), D702-R (SEQ ID No: 103), D703-R (SEQ ID No: 104), D704-R (SEQ ID No: 105), D705-R (SEQ ID No: 106), and D706-R (SEQ ID No: 107) which were shown in Table 18 as primers, PCR amplification products obtained through multiplex PCR, a multiplex PCR mix 1, a multiplex PCR mix 2, and water were mixed with each other to prepare a reaction solution.

After performing initial thermal denaturation for 3 minutes at 94° C. using each of the prepared reaction solution, a thermal cycle of thermal denaturation performed for 30 seconds at 94° C., annealing performed for 60 seconds at 50° C., and an elongation reaction performed for 30 seconds at 72° C. was performed 5 cycles and a thermal cycle of thermal denaturation performed for 45 seconds at 94° C., annealing performed for 60 seconds at 55° C., and an elongation reaction performed for 30 seconds at 72° C. was further performed 11 cycles. The above-described multiplex PCR mix 1 and the above-described multiplex PCR mix 2 are reagents contained in MULTIPLEX PCR ASSAY KIT (manufactured by TAKARA BIO INC.).

TABLE 18

| | | SEQ |
|---|---|---|
| Primer | | ID |
| name | Base sequence (5′ → 3′) | No |
| D501-F | AATGATACGGCGACCACCGAGATCTACACTATAGCCTTC | 101 |
| | TTTCCCTACACGACGCTCTTCCGATCTCTG | |
| D701-R | CAAGCAGAAGACGGCATACGAGATCGAGTAATGTGACTG | 102 |
| | GAGTTCAGACGTGTGCTCTTCCGATCTGAC | |
| D702-R | CAAGCAGAAGACGGCATACGAGATTCTCCGGAGTGACTG | 103 |
| | GACTTCAGACGTGTGCTCTTCCGATCTGAC | |
| D703-R | GAAGCAGAAGAGGGCATAGGAGATAATGAGCGGTGACTG | 104 |
| | GAGTTCAGACGTGTGCTCTTCCGATCTGAC | |
| D704-R | CAAGCAGAAGACGGCATACGAGATGGAATCTCGTGACTG | 105 |
| | GAGTTCAGACGTGTGCTCTTCCGATCTGAC | |
| D705-R | CAAGCAGAAGACGGCATACGAGATTTCTGAATGTGACTG | 106 |
| | GAGTTCAGACGTGTGCTCTTCCGATCTGAC | |
| D706-R | CAAGCAGAAGACGGCATACGAGATACGAATTCGTGACTG | 107 |
| | GAGTTCAGACGTGTGCTCTTCCGATCTGAC | |

The PCR amplification products obtained through multiplex PCR were purified using DNA purification reagent kit AMPure XP (manufactured by Beckman Coulter Inc.) and the concentrations thereof were measured using Agilent 2100 BIOANALYZER (manufactured by Agilent Technologies).

Quantitative determination was performed as more accurate quantitative determination of amplification products using KAPA LIBRARY QUANTIFICATION KIT (manufactured by NIPPON Genetics Co, Ltd.).

(Measurement of Number of Times of Sequence Reading (Coverage, Sequence Depth))

The coverage (sequence depth) for each target locus of chromosome 13, chromosome 18, and chromosome 21 of nucleated red blood cells which were identified as being derived from a fetus was measured by performing sequencing of amplification products using a next generation sequencer MiSeq (registered trademark manufactured by Illumina, Inc.). The amount of amplification products (number of times of sequence reading) of target loci of chromosome 21 of nucleated cells which were identified as being derived from a mother were separately measured by performing sequencing of the amplification products using Miseq.

The coverage (sequence depth) was calculated for each locus and the variation in coverage was evaluated using the coefficient of variation (CV). As a result, the coefficient of variation was about 6.5% which indicates small variation in coverage.

### Comparative Example 1

Although the point that chromosomes of interest are chromosome 13, chromosome 18, and chromosome 21 is not different from that of Example 1, the number of loci on chromosome 13 was set to 75, the number of loci on chromosome 18 was set to 77, the number of loci on chromosome 21 was set to 76, the number of loci on an X chromosome was set to 34, and the number of loci on a Y chromosome was set to 20, so that the total number of loci became 282.

The coverage was calculated for each locus while setting the other conditions to be the same as those in Example 1, and the variation in coverage was evaluated using the coefficient of variation. As a result, the coefficient of variation was about 28.2% which indicates large variation in coverage.

### Comparative Example 2

Although the point that chromosomes of interest are chromosome 13, chromosome 18, and chromosome 21 is not different from that of Example 1, the number of loci on chromosome 13 was set to 52, the number of loci on chromosome 18 was set to 49, the number of loci on chromosome 21 was set to 46, the number of loci on an X chromosome was set to 34, and the number of loci on a Y chromosome was set to 20, so that the total number of loci became 201.

The coverage was calculated for each locus while setting the other conditions to be the same as those in Example 1, and the variation in coverage was evaluated using the coefficient of variation. As a result, the coefficient of variation was about 32.7% which indicates large variation in coverage.

### Comparative Example 3

Although the point that chromosomes of interest are chromosome 13, chromosome 18, and chromosome 21 is not different from that of Example 1, the number of loci on chromosome 13 was set to 20, the number of loci on chromosome 18 was set to 20, the number of loci on chromosome 21 was set to 20, the number of loci on an X chromosome was set to 20, and the number of loci on a Y chromosome was set to 20, so that the total number of loci became 100.

The coverage was calculated for each locus while setting the other conditions to be the same as those in Example 1, and the variation in coverage was evaluated using the coefficient of variation. As a result, the coefficient of variation was about 52.9% which indicates large variation in coverage.

### Comparative Example 4

Although the point that chromosomes of interest are chromosome 13, chromosome 18, and chromosome 21 is not different from that of Example 1, the number of loci on chromosome 13 was set to 9, the number of loci on chromosome 18 was set to 9, the number of loci on chromosome 21 was set to 8, and the number of loci on an X chromosome was set to 9, so that the total number of loci became 35.

The coverage was calculated for each locus while setting the other conditions to be the same as those in Example 1, and the variation in coverage was evaluated using the coefficient of variation. As a result, the coefficient of variation was about 143.1% which indicates large variation in coverage.

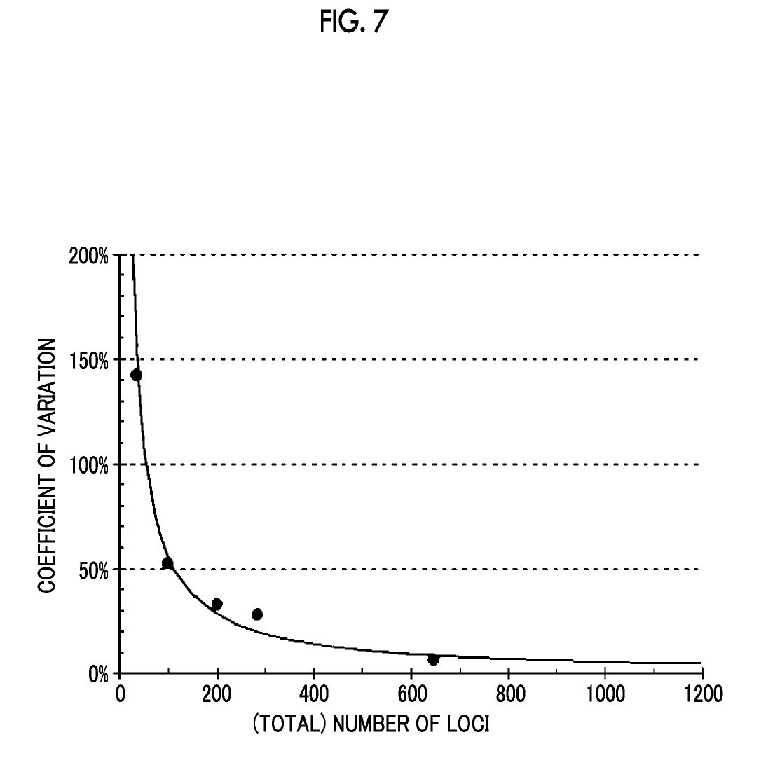

FIG. 7 shows a graph in which the (total) number of loci is plotted on the lateral axis and the coefficient of variation of the coverage is plotted on the longitudinal axis. The plots correspond to Example 1 and Comparative Examples 1 to 4, and the curve is an approximate curve obtained from the plots.

In a case where the number of loci on chromosomes of interest is set to be greater than or equal to 80, the coefficient of variation of the coverage becomes sufficiently small and the variation of coverage for each locus is reduced. Therefore, it is considered that it is possible to accurately perform quantitative determination of the number of chromosomes from a small amount of DNA of a single cell, a small number of cells, or the like.

### Sequence List

International Application W-5931PCT Method for Determining Number of Chromosomes JP17004390 20170207-00260357151700267697 Normal 20170207093843201701120942231730_P1AP101_W-_18.app Based on International Patent Cooperation Treaty

What is claimed is:

1. A chromosome number determination method of chromosomes of interest, comprising:
a step of performing multiplex PCR for simultaneously amplifying a plurality of loci on the chromosomes using genomic DNA extracted from a single cell or a small number of cells as templates,
wherein the number of loci on the chromosomes of interest is greater than or equal to 80 per chromosome,
wherein a plurality of primer sets used in the multiplex PCR are designed through a method for designing primer sets used in the polymerase chain reaction, the method for designing primer sets including
a target locus selection step of selecting a target locus for designing primer sets used in the multiplex PCR from the plurality of loci,
a primer candidate base sequence generation step of generating at least one base sequence of a primer candidate for amplifying the target locus regarding each of a forward-side primer and a reverse-side primer based on a base sequence in a vicinity region of the target locus on the chromosomes,
a local alignment step of obtaining a local alignment score by performing pairwise local alignment on the base sequence of the primer candidate for amplifying the target locus under a condition that a partial sequence to be subjected to comparison includes the 3′ terminal of the base sequence of the primer candidate for amplifying the target locus,
a first stage selection step of performing first stage selection of the base sequence of the primer candidate for amplifying the target locus based on the local alignment score obtained in the local alignment step,
a global alignment step of obtaining a global alignment score by performing pairwise global alignment on a base sequence which has a predetermined sequence length and includes the 3′ terminal of the base sequence of the primer candidate for amplifying the target locus,
a second stage selection step of performing second stage selection of the base sequence of the primer candidate for amplifying the target locus based on the global alignment score obtained in the global alignment step, and
a primer employment step of employing the base sequence of the primer candidate which has been selected in both of the first stage selection step and the second stage selection step as the base sequence of the primer for amplifying the target locus, and
wherein both steps of the local alignment step and the first stage selection step are performed before or after both steps of the global alignment step and the second stage selection step, or performed in parallel with both steps of the global alignment step and the second stage selection step.

2. The chromosome number determination method according to claim 1,
wherein the number of loci on the chromosomes of interest is 80 to 1,000 per chromosome.

3. The chromosome number determination method according to claim 1,
wherein the number of loci on the chromosomes of interest is 100 to 1,000 per chromosome.

4. The chromosome number determination method according to claim 1,
wherein the number of loci on the chromosomes of interest is 100 to 500 per chromosome.

5. The chromosome number determination method according to claim 1,
wherein the chromosomes of interest contain at least one selected from the group consisting of chromosome 13, chromosome 18, and chromosome 21.

6. The chromosome number determination method according to claim 1,
wherein the steps from the target locus selection step to the primer employment step are repeated until the primer sets used in the multiplex PCR are employed for all of the plurality of loci.

7. The chromosome number determination method according to claim 1,
wherein one or more loci are selected in the target locus selection step.

8. The chromosome number determination method according to claim 1,
wherein primer sets used in the multiplex PCR are designed through a method for designing primer sets used in the polymerase chain reaction, the method for designing primer sets including
a first target locus selection step of selecting a first target locus for designing primer sets used in the multiplex PCR from the plurality of loci,
a first primer candidate base sequence generation step of generating at least one base sequence of a primer candidate for amplifying the first target locus regarding each of a forward-side primer and a reverse-side primer based on a base sequence in a vicinity region of the first target locus on the chromosomes,
a first local alignment step of obtaining a local alignment score by performing pairwise local alignment on the base sequence of the primer candidate for amplifying the first target locus under a condition that a partial sequence to be subjected to comparison includes the 3′ terminal of the base sequence of the primer candidate for amplifying the first target locus,
a first step of first stage selection of performing first stage selection of the base sequence of the primer candidate for amplifying the first target locus based on the local alignment score obtained in the first local alignment step,
a first global alignment step of obtaining a global alignment score by performing pairwise global alignment on a base sequence which has a predetermined sequence length and includes the 3′ terminal of the base sequence of the primer candidate for amplifying the first target locus,
a first step of second stage selection of performing second stage selection of the base sequence of the primer candidate for amplifying the first target locus based on the global alignment score obtained in the first global alignment step,
a first primer employment step of employing the base sequence of the primer candidate which has been selected in both of the first step of first stage selection and the first step of second stage selection as a base sequence of a primer for amplifying the first target locus,
a second target locus selection step of selecting a second target locus, which is different from the already selected target locus and in which primer sets used in the multiplex PCR are designed, from the plurality of loci,
a second primer candidate base sequence generation step of generating at least one base sequence of a primer candidate for amplifying the second target locus regarding each of a forward-side primer and a reverse-side primer based on a base sequence in a vicinity region of the second target locus on the chromosomes,
a second local alignment step of obtaining a local alignment score by performing pairwise local alignment on the base sequence of the primer candidate for amplifying the second target locus and the base sequence of the primer which has already been employed, under a condition that partial sequences to be subjected to comparison include the 3′ terminal of the base sequence of the primer candidate for amplifying the second target locus and the 3′ terminal of the base sequence of the primer which has already been employed,
a second step of first stage selection of performing first stage selection of the base sequence of the primer candidate for amplifying the second target locus based on the local alignment score obtained in the second local alignment step,
a second global alignment step of obtaining a global alignment score by performing pairwise global alignment on base sequences which have a predetermined sequence length and include the 3′ terminal of the base sequence of the primer candidate for amplifying the second target locus and the 3′ terminal of the base sequence of the primer which has already been employed,
a second step of second stage selection of performing second stage selection of the base sequence of the primer candidate for amplifying the second target locus based on the global alignment score obtained in the second global alignment step, and
a second primer employment step of employing the base sequence of the primer candidate which has been selected in both of the second step of first stage selection and the second step of second stage selection as a base sequence of a primer for amplifying the second target locus,
wherein both steps of the first local alignment step and the first step of first stage selection are performed before or after both steps of the first global alignment step and the first step of second stage selection, or performed in parallel with both steps of the first global alignment step and the first step of second stage selection,
wherein both steps of the second local alignment step and the second step of first stage selection are performed before or after both steps of the second global alignment step and the second step of second stage selection, or performed in parallel with both steps of the second global alignment step and the second step of second stage selection, and
wherein, in a case where the number of the plurality of loci is three or more, the steps from the second target locus selection step to the second primer employment step are repeated until the primer sets used in the multiplex PCR are employed for all of the plurality of loci.

9. The chromosome number determination method according to claim 2,
wherein the number of loci on the chromosomes of interest is 100 to 1,000 per chromosome.

10. The chromosome number determination method according to claim 2,
wherein the number of loci on the chromosomes of interest is 100 to 500 per chromosome.

11. The chromosome number determination method according to claim 2,
wherein the chromosomes of interest contain at least one selected from the group consisting of chromosome 13, chromosome 18, and chromosome 21.

12. The chromosome number determination method according to claim 2,
wherein the steps from the target locus selection step to the primer employment step are repeated until the primer sets used in the multiplex PCR are employed for all of the plurality of loci.

13. The chromosome number determination method according to claim 2,
wherein one or more loci are selected in the target locus selection step.

14. The chromosome number determination method according to claim 2,
wherein primer sets used in the multiplex PCR are designed through a method for designing primer sets used in the polymerase chain reaction, the method for designing primer sets including
a first target locus selection step of selecting a first target locus for designing primer sets used in the multiplex PCR from the plurality of loci,
a first primer candidate base sequence generation step of generating at least one base sequence of a primer candidate for amplifying the first target locus regarding each of a forward-side primer and a reverse-side primer based on a base sequence in a vicinity region of the first target locus on the chromosomes,
a first local alignment step of obtaining a local alignment score by performing pairwise local alignment on the base sequence of the primer candidate for amplifying the first target locus under a condition that a partial sequence to be subjected to comparison includes the 3′ terminal of the base sequence of the primer candidate for amplifying the first target locus,
a first step of first stage selection of performing first stage selection of the base sequence of the primer candidate for amplifying the first target locus based on the local alignment score obtained in the first local alignment step,
a first global alignment step of obtaining a global alignment score by performing pairwise global alignment on a base sequence which has a predetermined sequence length and includes the 3′ terminal of the base sequence of the primer candidate for amplifying the first target locus,
a first step of second stage selection of performing second stage selection of the base sequence of the primer candidate for amplifying the first target locus based on the global alignment score obtained in the first global alignment step,
a first primer employment step of employing the base sequence of the primer candidate which has been selected in both of the first step of first stage selection and the first step of second stage selection as a base sequence of a primer for amplifying the first target locus,
a second target locus selection step of selecting a second target locus, which is different from the already selected target locus and in which primer sets used in the multiplex PCR are designed, from the plurality of loci,
a second primer candidate base sequence generation step of generating at least one base sequence of a primer candidate for amplifying the second target locus regarding each of a forward-side primer and a reverse-side primer based on a base sequence in a vicinity region of the second target locus on the chromosomes,
a second local alignment step of obtaining a local alignment score by performing pairwise local alignment on the base sequence of the primer candidate for amplifying the second target locus and the base sequence of the primer which has already been employed, under a condition that partial sequences to be subjected to comparison include the 3′ terminal of the base sequence of the primer candidate for amplifying the second target locus and the 3′ terminal of the base sequence of the primer which has already been employed,
a second step of first stage selection of performing first stage selection of the base sequence of the primer candidate for amplifying the second target locus based on the local alignment score obtained in the second local alignment step,
a second global alignment step of obtaining a global alignment score by performing pairwise global alignment on base sequences which have a predetermined sequence length and include the 3′ terminal of the base sequence of the primer candidate for amplifying the second target locus and the 3′ terminal of the base sequence of the primer which has already been employed,
a second step of second stage selection of performing second stage selection of the base sequence of the primer candidate for amplifying the second target locus based on the global alignment score obtained in the second global alignment step, and
a second primer employment step of employing the base sequence of the primer candidate which has been selected in both of the second step of first stage selection and the second step of second stage selection as a base sequence of a primer for amplifying the second target locus,
wherein both steps of the first local alignment step and the first step of first stage selection are performed before or after both steps of the first global alignment step and the first step of second stage selection, or performed in parallel with both steps of the first global alignment step and the first step of second stage selection,
wherein both steps of the second local alignment step and the second step of first stage selection are performed before or after both steps of the second global alignment step and the second step of second stage selection, or performed in parallel with both steps of the second global alignment step and the second step of second stage selection, and
wherein, in a case where the number of the plurality of loci is three or more, the steps from the second target locus selection step to the second primer employment step are repeated until the primer sets used in the multiplex PCR are employed for all of the plurality of loci.

15. The chromosome number determination method according to claim 3,
wherein the number of loci on the chromosomes of interest is 100 to 500 per chromosome.

16. The chromosome number determination method according to claim 3,
wherein the chromosomes of interest contain at least one selected from the group consisting of chromosome 13, chromosome 18, and chromosome 21.

17. The chromosome number determination method according to claim 3,
wherein the steps from the target locus selection step to the primer employment step are repeated until the primer sets used in the multiplex PCR are employed for all of the plurality of loci.

18. The chromosome number determination method according to claim 3,
wherein one or more loci are selected in the target locus selection step.

19. The chromosome number determination method according to claim 3,
wherein primer sets used in the multiplex PCR are designed through a method for designing primer sets used in the polymerase chain reaction, the method for designing primer sets including
a first target locus selection step of selecting a first target locus for designing primer sets used in the multiplex PCR from the plurality of loci,
a first primer candidate base sequence generation step of generating at least one base sequence of a primer candidate for amplifying the first target locus regarding each of a forward-side primer and a reverse-side primer based on a base sequence in a vicinity region of the first target locus on the chromosomes,
a first local alignment step of obtaining a local alignment score by performing pairwise local alignment on the base sequence of the primer candidate for amplifying the first target locus under a condition that a partial sequence to be subjected to comparison includes the 3′ terminal of the base sequence of the primer candidate for amplifying the first target locus,
a first step of first stage selection of performing first stage selection of the base sequence of the primer candidate for amplifying the first target locus based on the local alignment score obtained in the first local alignment step,
a first global alignment step of obtaining a global alignment score by performing pairwise global alignment on a base sequence which has a predetermined sequence length and includes the 3′ terminal of the base sequence of the primer candidate for amplifying the first target locus,
a first step of second stage selection of performing second stage selection of the base sequence of the primer candidate for amplifying the first target locus based on the global alignment score obtained in the first global alignment step,
a first primer employment step of employing the base sequence of the primer candidate which has been selected in both of the first step of first stage selection and the first step of second stage selection as a base sequence of a primer for amplifying the first target locus,
a second target locus selection step of selecting a second target locus, which is different from the already selected target locus and in which primer sets used in the multiplex PCR are designed, from the plurality of loci,
a second primer candidate base sequence generation step of generating at least one base sequence of a primer candidate for amplifying the second target locus regarding each of a forward-side primer and a reverse-side primer based on a base sequence in a vicinity region of the second target locus on the chromosomes,
a second local alignment step of obtaining a local alignment score by performing pairwise local alignment on the base sequence of the primer candidate for amplifying the second target locus and the base sequence of the primer which has already been employed, under a condition that partial sequences to be subjected to comparison include the 3′ terminal of the base sequence of the primer candidate for amplifying the second target locus and the 3′ terminal of the base sequence of the primer which has already been employed,
a second step of first stage selection of performing first stage selection of the base sequence of the primer candidate for amplifying the second target locus based on the local alignment score obtained in the second local alignment step,
a second global alignment step of obtaining a global alignment score by performing pairwise global alignment on base sequences which have a predetermined sequence length and include the 3′ terminal of the base sequence of the primer candidate for amplifying the second target locus and the 3′ terminal of the base sequence of the primer which has already been employed,
a second step of second stage selection of performing second stage selection of the base sequence of the primer candidate for amplifying the second target locus based on the global alignment score obtained in the second global alignment step, and
a second primer employment step of employing the base sequence of the primer candidate which has been selected in both of the second step of first stage selection and the second step of second stage selection as a base sequence of a primer for amplifying the second target locus,
wherein both steps of the first local alignment step and the first step of first stage selection are performed before or after both steps of the first global alignment step and the first step of second stage selection, or performed in parallel with both steps of the first global alignment step and the first step of second stage selection,
wherein both steps of the second local alignment step and the second step of first stage selection are performed before or after both steps of the second global alignment step and the second step of second stage selection, or performed in parallel with both steps of the second global alignment step and the second step of second stage selection, and
wherein, in a case where the number of the plurality of loci is three or more, the steps from the second target locus selection step to the second primer employment step are repeated until the primer sets used in the multiplex PCR are employed for all of the plurality of loci.

20. The chromosome number determination method according to claim 4,
wherein primer sets used in the multiplex PCR are designed through a method for designing primer sets used in the polymerase chain reaction, the method for designing primer sets including
a first target locus selection step of selecting a first target locus for designing primer sets used in the multiplex PCR from the plurality of loci,
a first primer candidate base sequence generation step of generating at least one base sequence of a primer candidate for amplifying the first target locus regarding each of a forward-side primer and a reverse-side primer based on a base sequence in a vicinity region of the first target locus on the chromosomes,
a first local alignment step of obtaining a local alignment score by performing pairwise local alignment on the base sequence of the primer candidate for amplifying the first target locus under a condition that a partial sequence to be subjected to comparison includes the 3′ terminal of the base sequence of the primer candidate for amplifying the first target locus,
a first step of first stage selection of performing first stage selection of the base sequence of the primer candidate for amplifying the first target locus based on the local alignment score obtained in the first local alignment step,
a first global alignment step of obtaining a global alignment score by performing pairwise global alignment on a base sequence which has a predetermined sequence length and includes the 3′ terminal of the base sequence of the primer candidate for amplifying the first target locus,
a first step of second stage selection of performing second stage selection of the base sequence of the primer candidate for amplifying the first target locus based on the global alignment score obtained in the first global alignment step,
a first primer employment step of employing the base sequence of the primer candidate which has been selected in both of the first step of first stage selection and the first step of second stage selection as a base sequence of a primer for amplifying the first target locus,
a second target locus selection step of selecting a second target locus, which is different from the already selected target locus and in which primer sets used in the multiplex PCR are designed, from the plurality of loci,
a second primer candidate base sequence generation step of generating at least one base sequence of a primer candidate for amplifying the second target locus regarding each of a forward-side primer and a reverse-side primer based on a base sequence in a vicinity region of the second target locus on the chromosomes,
a second local alignment step of obtaining a local alignment score by performing pairwise local alignment on the base sequence of the primer candidate for amplifying the second target locus and the base sequence of the primer which has already been employed, under a condition that partial sequences to be subjected to comparison include the 3′ terminal of the base sequence of the primer candidate for amplifying the second target locus and the 3′ terminal of the base sequence of the primer which has already been employed,
a second step of first stage selection of performing first stage selection of the base sequence of the primer candidate for amplifying the second target locus based on the local alignment score obtained in the second local alignment step,
a second global alignment step of obtaining a global alignment score by performing pairwise global alignment on base sequences which have a predetermined sequence length and include the 3′ terminal of the base sequence of the primer candidate for amplifying the second target locus and the 3′ terminal of the base sequence of the primer which has already been employed,
a second step of second stage selection of performing second stage selection of the base sequence of the primer candidate for amplifying the second target locus based on the global alignment score obtained in the second global alignment step, and
a second primer employment step of employing the base sequence of the primer candidate which has been selected in both of the second step of first stage selection and the second step of second stage selection as a base sequence of a primer for amplifying the second target locus,
wherein both steps of the first local alignment step and the first step of first stage selection are performed before or after both steps of the first global alignment step and the first step of second stage selection, or performed in parallel with both steps of the first global alignment step and the first step of second stage selection,
wherein both steps of the second local alignment step and the second step of first stage selection are performed before or after both steps of the second global alignment step and the second step of second stage selection, or performed in parallel with both steps of the second global alignment step and the second step of second stage selection, and
wherein, in a case where the number of the plurality of loci is three or more, the steps from the second target locus selection step to the second primer employment step are repeated until the primer sets used in the multiplex PCR are employed for all of the plurality of loci.


SEQUENCE LISTING (107 sequences)

SEQ ID NO: 1 - DNA, 37 bp, Artificial Sequence
  chr13 20763380 Fwd
cgctcttccgatctctgcttcgatgcggaccttctgg

SEQ ID NO: 2 - DNA, 37 bp, Artificial Sequence
  chr13 20763380 Rev
cgctcttccgatctgactctcccacatccggctatgg

SEQ ID NO: 3 - DNA, 37 bp, Artificial Sequence
  chr13 21205192 Fwd
cgctcttccgatctctgtttccccgaccataagcttg

SEQ ID NO: 4 - DNA, 37 bp, Artificial Sequence
  chr13 21205192 Rev
cgctcttccgatctgacatacagggctgagagattgg

SEQ ID NO: 5 - DNA, 37 bp, Artificial Sequence
  chr13 21620085 Fwd
cgctcttccgatctctgtgataaggtccgaactttgg

SEQ ID NO: 6 - DNA, 37 bp, Artificial Sequence
  chr13 21620085 Rev
cgctcttccgatctgacgcgactgcaagagattcgtg

SEQ ID NO: 7 - DNA, 37 bp, Artificial Sequence
  chr13 23898509 Fwd
cgctcttccgatctctgatttgctgctgaccagggtg

SEQ ID NO: 8 - DNA, 37 bp, Artificial Sequence
  chr13 23898509 Rev
cgctcttccgatctgacaggtacagcttcccatctgg

SEQ ID NO: 9 - DNA, 37 bp, Artificial Sequence
  chr13 24797913 Fwd
cgctcttccgatctctgccgtgtgtgagattctcgtg

SEQ ID NO: 10 - DNA, 37 bp, Artificial Sequence
  chr13 24797913 Rev
cgctcttccgatctgacactgctcagggtcctctgtg

SEQ ID NO: 11 - DNA, 37 bp, Artificial Sequence
  chr13 25009099 Fwd
cgctcttccgatctctggtaaagcctccaggatgttg

SEQ ID NO: 12 - DNA, 37 bp, Artificial Sequence
  chr13 25009099 Rev
cgctcttccgatctgacctggcacttgtgctgactgg

SEQ ID NO: 13 - DNA, 37 bp, Artificial Sequence
  chr13 25029218 Fwd
cgctcttccgatctctgccaaagcgcactcacctgtg

SEQ ID NO: 14 - DNA, 37 bp, Artificial Sequence
  chr13 25029218 Rev
cgctcttccgatctgactagccagtgagagcgaagtg

SEQ ID NO: 15 - DNA, 37 bp, Artificial Sequence
  chr13 25265103 Fwd
cgctcttccgatctctgggcctagaggacgatgcttg

SEQ ID NO: 16 - DNA, 37 bp, Artificial Sequence
  chr13 25265103 Rev
cgctcttccgatctgactgttgataaccatgccggtg

SEQ ID NO: 17 - DNA, 37 bp, Artificial Sequence
  chr13 25266932 Fwd
cgctcttccgatctctgtgctggacagtgactcatgg

SEQ ID NO: 18 - DNA, 37 bp, Artificial Sequence
  chr13 25266932 Rev
cgctcttccgatctgaccattttcctgtcctggcttg

SEQ ID NO: 19 - DNA, 37 bp, Artificial Sequence
  chr13 25453420 Fwd
cgctcttccgatctctgatccagttcatatgccgttg

SEQ ID NO: 20 - DNA, 37 bp, Artificial Sequence
  chr13 25453420 Rev
cgctcttccgatctgacgcgttgctgtcattcctttg

SEQ ID NO: 21 - DNA, 37 bp, Artificial Sequence
  chr18 346821 Fwd
cgctcttccgatctctgaggttctgctcgttggcttg

SEQ ID NO: 22 - DNA, 37 bp, Artificial Sequence
  chr18 346821 Rev
cgctcttccgatctgacccaagaaggacacggattgg

SEQ ID NO: 23 - DNA, 37 bp, Artificial Sequence
  chr18 3075712 Fwd
cgctcttccgatctctgagcttcccgggaaattagtg

SEQ ID NO: 24 - DNA, 37 bp, Artificial Sequence
  chr18 3075712 Rev
cgctcttccgatctgacgaattttaatcgcccctgtg

SEQ ID NO: 25 - DNA, 37 bp, Artificial Sequence
  chr18 3188976 Fwd
cgctcttccgatctctgggactgcttagatgccgtgg

SEQ ID NO: 26 - DNA, 37 bp, Artificial Sequence
  chr18 3188976 Rev
cgctcttccgatctgacagtcaaaagcatgtcagtgg

SEQ ID NO: 27 - DNA, 37 bp, Artificial Sequence
  chr18 3457539 Fwd
cgctcttccgatctctgctctgtggagtccgtgatgg

SEQ ID NO: 28 - DNA, 37 bp, Artificial Sequence
  chr18 3457539 Rev
cgctcttccgatctgacatgcagtcacagtggtatgg

SEQ ID NO: 29 - DNA, 37 bp, Artificial Sequence
  chr18 5416160 Fwd
cgctcttccgatctctgggtaatgctgcaagctctgg

SEQ ID NO: 30 - DNA, 37 bp, Artificial Sequence
  chr18 5416160 Rev
cgctcttccgatctgaccctaggggatcaagatgtgg

SEQ ID NO: 31 - DNA, 37 bp, Artificial Sequence
  chr18 5956238 Fwd
cgctcttccgatctctgattcatcccctgacttcttg

SEQ ID NO: 32 - DNA, 37 bp, Artificial Sequence
  chr18 5956238 Rev
cgctcttccgatctgactatttgcagcagatcgatgg

SEQ ID NO: 33 - DNA, 37 bp, Artificial Sequence
  chr18 6943264 Fwd
cgctcttccgatctctgcaccaacataaatgggattg

SEQ ID NO: 34 - DNA, 37 bp, Artificial Sequence
  chr18 6943264 Rev
cgctcttccgatctgacgccactgtgctctgtgatgg

SEQ ID NO: 35 - DNA, 37 bp, Artificial Sequence
  chr18 6983194 Fwd
cgctcttccgatctctgagtcctgtgagcatctctgg

SEQ ID NO: 36 - DNA, 37 bp, Artificial Sequence
  chr18 6983194 Rev
cgctcttccgatctgactgagtgaattggcaagtttg

SEQ ID NO: 37 - DNA, 37 bp, Artificial Sequence
  chr18 8796223 Fwd
cgctcttccgatctctggcagtttgcagggactcttg

SEQ ID NO: 38 - DNA, 37 bp, Artificial Sequence
  chr18 8796223 Rev
cgctcttccgatctgaccactccaggttcgctcatgg

SEQ ID NO: 39 - DNA, 37 bp, Artificial Sequence
  chr18 8798185 Fwd
cgctcttccgatctctgctcctgcctgtttcagggtg

SEQ ID NO: 40 - DNA, 37 bp, Artificial Sequence
  chr18 8798185 Rev
cgctcttccgatctgacaatctgcacccgggagtgtg

SEQ ID NO: 41 - DNA, 37 bp, Artificial Sequence
  chr21 16340289 Fwd
cgctcttccgatctctggcaacagtctggcttttttg

SEQ ID NO: 42 - DNA, 37 bp, Artificial Sequence
  chr21 16340289 Rev
cgctcttccgatctgacgggatcaggtactgccgttg

SEQ ID NO: 43 - DNA, 37 bp, Artificial Sequence
  chr21 28212760 Fwd
cgctcttccgatctctgcaaggtgctacatgtgctgg

SEQ ID NO: 44 - DNA, 37 bp, Artificial Sequence
  chr21 28212760 Rev
cgctcttccgatctgaccctttgcaggggaatgtttg

SEQ ID NO: 45 - DNA, 37 bp, Artificial Sequence
  chr21 28305212 Fwd
cgctcttccgatctctggagcagcgtaccattgggtg

SEQ ID NO: 46 - DNA, 37 bp, Artificial Sequence
  chr21 28305212 Rev
cgctcttccgatctgaccagtgttctcgctcatgtgg

SEQ ID NO: 47 - DNA, 37 bp, Artificial Sequence
  chr21 30408670 Fwd
cgctcttccgatctctgtgtctccccctttttagttg

SEQ ID NO: 48 - DNA, 37 bp, Artificial Sequence
  chr21 30408670 Rev
cgctcttccgatctgactttacctggctttggagttg

SEQ ID NO: 49 - DNA, 37 bp, Artificial Sequence
  chr21 30464866 Fwd
cgctcttccgatctctggtcagcaagttggctactgg

SEQ ID NO: 50 - DNA, 37 bp, Artificial Sequence
  chr21 30464866 Rev
cgctcttccgatctgacagccttaggctcccatggtg

SEQ ID NO: 51 - DNA, 37 bp, Artificial Sequence
  chr21 31709691 Fwd
cgctcttccgatctctgtgctgagtgctttctgattg

SEQ ID NO: 52 - DNA, 37 bp, Artificial Sequence
  chr21 31709691 Rev
cgctcttccgatctgactcacagacgatagctgtgtg

SEQ ID NO: 53 - DNA, 37 bp, Artificial Sequence
  chr21 31812772 Fwd
cgctcttccgatctctgcttctcctcctgctgttttg

SEQ ID NO: 54 - DNA, 37 bp, Artificial Sequence
  chr21 31812772 Rev
cgctcttccgatctgacccaaagtgcaggatgtctgg

SEQ ID NO: 55 - DNA, 37 bp, Artificial Sequence
  chr21 32253513 Fwd
cgctcttccgatctctggagactcctcccactggttg

SEQ ID NO: 56 - DNA, 37 bp, Artificial Sequence
  chr21 32253513 Rev
cgctcttccgatctgacaacccttgccaggtgacttg

SEQ ID NO: 57 - DNA, 37 bp, Artificial Sequence
  chr21 32638549 Fwd
cgctcttccgatctctgtcttcagcagcagctggtgg

SEQ ID NO: 58 - DNA, 37 bp, Artificial Sequence
  chr21 32638549 Rev
cgctcttccgatctgacccaattccttgggtgacttg

SEQ ID NO: 59 - DNA, 37 bp, Artificial Sequence
  chr21 33694120 Fwd
cgctcttccgatctctggcggaacccatgtacctgtg

SEQ ID NO: 60 - DNA, 37 bp, Artificial Sequence
  chr21 33694120 Rev
cgctcttccgatctgacaaacagagcagaagtgggtg

SEQ ID NO: 61 - DNA, 37 bp, Artificial Sequence
  chrX 2779570 Fwd
cgctcttccgatctctgagactcactccacgtgtgtg

SEQ ID NO: 62 - DNA, 37 bp, Artificial Sequence
  chrX 2779570 Rev
cgctcttccgatctgacagaacccagtggtgaatttg

SEQ ID NO: 63 - DNA, 37 bp, Artificial Sequence
  chrX 2951434 Fwd
cgctcttccgatctctgcttccccttctgtgggtgtg

SEQ ID NO: 64 - DNA, 37 bp, Artificial Sequence
  chrX 2951434 Rev
cgctcttccgatctgacaggataaaacaatgggttgg

SEQ ID NO: 65 - DNA, 37 bp, Artificial Sequence
  chrX 3241050 Fwd
cgctcttccgatctctgctgttgccgtctcttcatgg

SEQ ID NO: 66 - DNA, 37 bp, Artificial Sequence
  chrX 3241050 Rev
cgctcttccgatctgacacctctggaggaagttgttg

SEQ ID NO: 67 - DNA, 37 bp, Artificial Sequence
  chrX 6995417 Fwd
cgctcttccgatctctggaactccttgtggcggcttg

SEQ ID NO: 68 - DNA, 37 bp, Artificial Sequence
  chrX 6995417 Rev
cgctcttccgatctgaccctgcaagaaggtcttatgg

SEQ ID NO: 69 - DNA, 37 bp, Artificial Sequence
  chrX 14027177 Fwd
cgctcttccgatctctgctccatggcttggatcttgg

SEQ ID NO: 70 - DNA, 37 bp, Artificial Sequence
  chrX 14027177 Rev
cgctcttccgatctgacagcgcctggacagctatgtg

SEQ ID NO: 71 - DNA, 37 bp, Artificial Sequence
  chrX 16168467 Fwd
cgctcttccgatctctgtacgccaggtgtctcgcttg

SEQ ID NO: 72 - DNA, 37 bp, Artificial Sequence
  chrX 16168467 Rev
cgctcttccgatctgactccagataaaggcggctttg

SEQ ID NO: 73 - DNA, 37 bp, Artificial Sequence
  chrX 16627756 Fwd
cgctcttccgatctctggacagtttgcaaccctgttg

SEQ ID NO: 74 - DNA, 37 bp, Artificial Sequence
  chrX 16627756 Rev
cgctcttccgatctgactctgctttaatcgcatcgtg

SEQ ID NO: 75 - DNA, 37 bp, Artificial Sequence
  chrX 17746244 Fwd
cgctcttccgatctctgtgcaggtctggaggaagttg

SEQ ID NO: 76 - DNA, 37 bp, Artificial Sequence
  chrX 17746244 Rev
cgctcttccgatctgactaccaggcactttgtcatgg

SEQ ID NO: 77 - DNA, 37 bp, Artificial Sequence
  chrX 19375782 Fwd
cgctcttccgatctctgtaataaagggcctgcgtttg

SEQ ID NO: 78 - DNA, 37 bp, Artificial Sequence
  chrX 19375782 Rev
cgctcttccgatctgaccactcatactgtgtccgtgg

SEQ ID NO: 79 - DNA, 37 bp, Artificial Sequence
  chrX 22291606 Fwd
cgctcttccgatctctgagttaccagcgcttcgcttg

SEQ ID NO: 80 - DNA, 37 bp, Artificial Sequence
  chrX 22291606 Rev
cgctcttccgatctgacatgttgcacagacggtagtg

SEQ ID NO: 81 - DNA, 37 bp, Artificial Sequence
  chrY 2710802 Fwd
cgctcttccgatctctgaacaagggcaagaagttgtg

SEQ ID NO: 82 - DNA, 37 bp, Artificial Sequence
  chrY 2710802 Rev
cgctcttccgatctgaccaccatcaatgtggaaattg

SEQ ID NO: 83 - DNA, 37 bp, Artificial Sequence
  chrY 6740540 Fwd
cgctcttccgatctctgcttcagcacagatgttttgg

SEQ ID NO: 84 - DNA, 37 bp, Artificial Sequence
  chrY 6740540 Rev
cgctcttccgatctgacttttgtttgcctgccttgtg

SEQ ID NO: 85 - DNA, 37 bp, Artificial Sequence
  chrY 7262370 Fwd
cgctcttccgatctctgtgtcatcaatagttggctgg

SEQ ID NO: 86 - DNA, 37 bp, Artificial Sequence
  chrY 7262370 Rev
cgctcttccgatctgacagttcccttttgtagggtgg

SEQ ID NO: 87 - DNA, 37 bp, Artificial Sequence
  chrY 8624945 Fwd
cgctcttccgatctctgttgctgtgtgaagtccttgg

SEQ ID NO: 88 - DNA, 37 bp, Artificial Sequence
  chrY 8624945 Rev
cgctcttccgatctgacaagcggacagctgtgtctgg

SEQ ID NO: 89 - DNA, 37 bp, Artificial Sequence
  chrY 8632095 Fwd
cgctcttccgatctctgcctgggagctcgtgagtttg

SEQ ID NO: 90 - DNA, 37 bp, Artificial Sequence
  chrY 8632095 Rev
cgctcttccgatctgactcacgtctgcctagattttg

SEQ ID NO: 91 - DNA, 37 bp, Artificial Sequence
  chrY 14655058 Fwd
cgctcttccgatctctgcagagtatcaggccttctgg

SEQ ID NO: 92 - DNA, 37 bp, Artificial Sequence
  chrY 14655058 Rev
cgctcttccgatctgacggcttaccagcttgtagtgg

SEQ ID NO: 93 - DNA, 37 bp, Artificial Sequence
  chrY 14756402 Fwd
cgctcttccgatctctgccaaccatcacgaaaattgg

SEQ ID NO: 94 - DNA, 37 bp, Artificial Sequence
  chrY 14756402 Rev
cgctcttccgatctgactcgtctcgtactggagattg

SEQ ID NO: 95 - DNA, 37 bp, Artificial Sequence
  chrY 14944834 Fwd
cgctcttccgatctctgtgatactccaattgtggtgg

SEQ ID NO: 96 - DNA, 37 bp, Artificial Sequence
  chrY 14944834 Rev
cgctcttccgatctgacctgtgtttttctttgcggtg

SEQ ID NO: 97 - DNA, 37 bp, Artificial Sequence
  chrY 15872674 Fwd
cgctcttccgatctctgccccagtggacagagttttg

SEQ ID NO: 98 - DNA, 37 bp, Artificial Sequence
  chrY 15872674 Rev
cgctcttccgatctgacggagccaatgctgtgatgtg

SEQ ID NO: 99 - DNA, 37 bp, Artificial Sequence
  chrY 22902924 Fwd
cgctcttccgatctctgtgacattgaaggtagcgttg

SEQ ID NO: 100 - DNA, 37 bp, Artificial Sequence
  chrY 22902924 Rev
cgctcttccgatctgacgagaaatcggagttcattgg

SEQ ID NO: 101 - DNA, 69 bp, Artificial Sequence
  P5 sequence
aatgatacggcgaccaccgagatctacactatagccttctttccctacacgacgctcttccgatctctg

SEQ ID NO: 102 - DNA, 69 bp, Artificial Sequence
  P7 sequence-1
caagcagaagacggcatacgagatcgagtaatgtgactggagttcagacgtgtgctcttccgatctgac

SEQ ID NO: 103 - DNA, 69 bp, Artificial Sequence
  P7 sequence-2
caagcagaagacggcatacgagattctccggagtgactggagttcagacgtgtgctcttccgatctgac

SEQ ID NO: 104 - DNA, 69 bp, Artificial Sequence
  P7 sequence-3
caagcagaagacggcatacgagataatgagcggtgactggagttcagacgtgtgctcttccgatctgac

SEQ ID NO: 105 - DNA, 69 bp, Artificial Sequence
  P7 sequence-4
caagcagaagacggcatacgagatggaatctcgtgactggagttcagacgtgtgctcttccgatctgac

SEQ ID NO: 106 - DNA, 69 bp, Artificial Sequence
  P7 sequence-5
caagcagaagacggcatacgagatttctgaatgtgactggagttcagacgtgtgctcttccgatctgac

SEQ ID NO: 107 - DNA, 69 bp, Artificial Sequence
  P7 sequence-6
caagcagaagacggcatacgagatacgaattcgtgactggagttcagacgtgtgctcttccgatctgac